(19) United States
(12) Patent Application Publication
(10) Pub. No.: US 2009/0274687 A1
(43) Pub. Date: Nov. 5, 2009

(54) ATTENUATION OF HYPOXIA INDUCED CARDIOVASCULAR DISORDERS

(75) Inventors: Karen Young, Miami, FL (US); Cleide Suguihara, Miami, FL (US); Joshua M. Hare, Miami Beach, FL (US)

Correspondence Address: DARBY & DARBY P.C., P.O. BOX 770, Church Street Station, New York, NY, 10008-0770, US

(73) Assignee: University Of Miami, Miami, FL (US)

(21) Appl. No.: 12/434,833

(22) Filed: May 4, 2009

Related U.S. Application Data
(60) Provisional application No. 61/049,977, filed on May 2, 2008.

(51) Int. Cl. A61K 31/7088; A61K 38/02; A61K 39/395; A61P 11/00; C12N 5/00; C12Q 1/02

(52) U.S. Cl. 424/130.1; 514/44 R; 514/2; 435/375; 435/29

(57) ABSTRACT

Blockade of stromal derived factor-1 (SDF-1), a stem cell mobilizer and/or its receptor, chemokine receptor 4 (CXCR4) attenuates and reverses hypoxia-induced cardiopulmonary remodeling in vivo. Compositions for treating hypoxia-induced cardiovascular disorders modulate the SDF-1/CXCR4 axis.

### CROSS-REFERENCE TO RELATED APPLICATIONS

The present application claims the priority of U.S. provisional patent application No. 61/049,977 filed May 2, 2008, which is incorporated herein by reference in its entirety.

### FIELD OF THE INVENTION

The invention relates to the fields of biochemistry, pharmacology, and medicine. More particularly, the invention relates to methods and compositions for treatment of pulmonary hypertension and disorders thereof.

### BACKGROUND

Despite marked improvements in medical care, approximately 2 in 1000 neonates are often still perinatally exposed to intermittent or chronic periods of hypoxia. Indeed, unlike adults, the neonatal pulmonary vasculature response to chronic hypoxic exposure is much more rapid and severe. It results in derangement in the adaptation of the fetal circulation to one that supports postnatal life and contributes to the pathogenesis of diseases such as persistent pulmonary hypertension of the newborn, chronic lung disease of prematurity as well as congenital heart disease. It is typically characterized by profound proliferation of smooth muscle and adventitial cells in the pulmonary vasculature, and abnormal extension of smooth muscle into peripheral arteries along with impairment in alveolar development in preterm neonates.

### SUMMARY

This Summary is provided to present a summary of the invention to briefly indicate the nature and substance of the invention. It is submitted with the understanding that it will not be used to interpret or limit the scope or meaning of the claims.

Compositions comprise agents which modulate stromal derived factor-1 (SDF-1) expression and/or function, chemokine receptor 4 (CXCR4) expression and/or function. In some embodiments, the compositions comprise one or more other therapeutic agents.

Methods of preventing, reversing or treating patients with cardiovascular disorders associated with hypoxia comprise administration of one or more compositions comprising a modulator of SDF-1, and/or a modulator of CXCR4, and/or at least one other therapeutic agent, or combinations thereof. An example is the treatment of neonatal hypoxia-induced pulmonary hypertension, persistent pulmonary hypertension of the newborn as well as pulmonary hypertension of other etiologies such as sepsis.

### BRIEF DESCRIPTION OF THE DRAWINGS

FIGS. 1A, 1B are graphs showing that SDF-1 is up regulated in the lungs and right ventricles of neonatal mice with pulmonary hypertension. FIG. 1A: Neonatal mice with pulmonary hypertension following exposure to hypoxia for 1 or 2 wk (n=5-7 mice/group) had a 2 fold increase in SDF-1 protein expression as compared to normoxia (n=5-7 mice/group). *p<0.005 RA vs. Hyp 1 wk; ** p<0.005 RA vs. Hyp 2 wk. FIG. 1B: Neonatal mice with pressure overload induced RVH following exposure to hypoxia for 1 or 2 wk ((n =5-7 mice/group) had a 2 fold increase in SDF-1 protein expression as compared to normoxia. *p<0.0001, RA vs. Hyp 1 wk; ** p<0.0001, RA vs. Hyp 2 wk.

FIGS. 2A, 2B are scans of photographs showing that BM-derived cells migrate to the pulmonary vasculature during hypoxia-induced pulmonary hypertension. FIG. 2A shows confocal immunofluorescence images of pulmonary arterioles in control and pulmonary hypertensive mice demonstrating more EGFP⁺ BM-derived cells (green) in the pulmonary hypertensive mice as compared to control in which these are sparsely evident. Original Magnification×400. FIG. 2B: Immunohistochemistry demonstrating more EFP⁺ BM-derived cells (brown) predominantly in the adventitia and media of pulmonary arterioles of mice with pulmonary hypertension as compared to control. Original Magnification×400.

FIGS. 3A-3D show that progenitor cells are present in the lungs and right ventricles of neonatal mice with pulmonary hypertension. FIG. 3A shows representative confocal immunofluorescence images of lungs and right ventricles obtained from control and pulmonary hypertensive neonatal mice following staining with anti-c-kit (red) antibody and DAPI (blue). c-kit⁺ cells (red) were more abundantly present in the medial and adventitial layer of remodeled pulmonary arterioles as compared to control. Magnification×400. Scale Bar is 20 μm. FIG. 3B is a graph showing significantly increased c-kit⁺ cells/mm² in the lungs and right ventricles of neonatal mice with PH. The cells were quantified by confocal microscopy in 6 random×400 fields in a total of 5 mice/group (*Lung: RA vs. Hyp, p<0.03 and **RV: RA vs. Hyp, p<0.05). FIG. 3C shows representative confocal Immunofluorescence images of sections obtained from hypertrophied right ventricles which were double-stained with anti-c-kit (green), anti-Sca-1 (red), DAPI (blue), anti- GATA-4 or anti-Ki67 (white) demonstrating co-localization of c-kit and Sca-1 cells with GATA-4 and Ki-67. Magnification×400. Scale Bar is 10 μm. FIG. 3D shows Isl-1 cells (red) present in the hypertrophied right ventricles of neonatal mice with pulmonary hypertension. These cells co-localized with GATA-4 and Ki-67 (white). Magnification∴400. Scale Bar is 10 μm.

FIGS. 4A-4E are graphs showing that inhibition of the SDF-1/CXCR4 axis decreased stem cell marker expression in the lungs and right ventricles of neonatal mice with pulmonary hypertension. Neonatal mice with pulmonary hypertension had a marked increase in lung: FIG. 4A: c-kit (2 fold, *p<0.0001) and FIG. 4B: sca-1 (1.9 fold, *p<0.02)); FIG. 4C RV: c-kit (5 fold; *p<0.0001), FIG. 4D: RV sca-1(1.6 fold; *p<0.001) and FIG. 4E: RV Isl-1 (2 fold; *p<0.02)) expressions as compared to RA (n=5-7/group). As compared to Hyp PL mice, administration of anti-SDF-1 significantly decreased FIG. 4A) lung c-kit (** p<0.0008), FIG. 4B) lung Sca-1 (** p<0.02) and RV Sca-1 (** p<0.0005) expressions to near baseline values. Similarly, administration of AMD3100 significantly decreased FIG. 4A) lung c-kit (+p21 0.0001), FIG. 4B) lung Sca-1 (+p<0.006), FIG. 4C) RV c-kit (+p<0.01), FIG. 4D) RV Sca-1 (+p<0.0001) and FIG. 4E) RV Isl-1 (+p<0.05) expressions to near baseline values as compared to Hyp PL (n=5-7/group).

FIG. 5A-5I shows that the inhibition of the SDF-1/CXCR4 axis prevents and reverses pulmonary hypertension in neonatal hypoxic mice. FIG. 5A is a graph showing that administration of anti-SDF-1 antibody (n=19) or the CXCR4 antagonist AMD3100 (n=10) to neonatal mice exposed to hypoxia significantly attenuated RVSP as compared to placebo (n=19), (* p<0.0001 RA vs. Hyp PL, ** p<0.0002 Hyp PL vs. Hyp SDF, +p<0.00001 Hyp PL vs. Hyp AMD). FIG. 5B is a graph showing that administration of anti-SDF-1 antibody (n=14) or the CXCR4 antagonist AMD3100 (n=6) significantly attenuated RV Hypertrophy as compared to placebo (n=14), (* p<0.001 RA vs. Hyp PL,** p<0.005 Hyp PL vs. Hyp SDF, +p<0.008 Hyp PL vs. Hyp AMD). FIG. 5C shows representative bright-field photomicrographs of lung sections stained with α-smooth muscle actin demonstrating decreased number of muscularized pulmonary arterioles in AMD3100 treated mice as compared to placebo. Non-muscularized, partially-muscularized and fully muscularized were defined as a-smooth muscle actin staining 0-25%, 25-75% and >75% of vessel circumference respectively. Magnification×200. FIG. 5D is a graph showing that morphometric analysis performed on 35-40 (20-50 μm diameter) pulmonary arterioles (n=5/group) demonstrated that AMD3100 markedly attenuated vascular remodeling (* p<0.04 RA vs. Hyp PL; ** p<0.05 Hyp PL vs. Hyp AMD). FIG. 5E is a graph showing that the percentage of non-muscularized pulmonary arterioles was significantly increased in the hypoxia treated mice as compared to placebo (* p21 0.001 RA vs. Hyp PL; ** p<0.04 Hyp PL vs. Hyp AMD; n=5/group). FIG. 5F is a graph showing that the mean linear intercept was significantly decreased in the hypoxia treated mice as compared to placebo (* p<0.005 RA vs. Hyp PL; ** p<0.05 Hyp PL vs. Hyp AMD; n=5/group). FIG. 5G is a graph showing that administration of the CXCR4 antagonist AMD3100 (n=7) to mice with established PH, significantly attenuated RVSP as compared to placebo, (* p<0.0003 RA vs. Hyp PL, ** p<0.007 Hyp PL vs. Hyp AMD). FIG. 5H is a graph showing that exposure of neonatal mice to hypoxia (n32 6) for 2 wk resulted in a marked RV Hypertrophy as compared to normoxia (n=8), (* p<0.0001 RA vs. Hyp PL), but this was not reversed following administration of AMD3100. FIG. 5I is a graph showing that the morphometric analysis demonstrated that the percentage of non-muscular pulmonary arterioles was significantly increased in the hypoxia treated mice as compared to placebo (* p<0.03 RA vs. Hyp PL; ** p<0.05 Hyp PL vs. Hyp AMD; n=4/group).

FIGS. 6A-6E show that the inhibition of SDF-1/CXCR4 axis decreases pulmonary vascular proliferation and apoptosis. FIG. 6A are representative immunofluorescence images of lungs obtained from RA, HYP PL and HYP AMD neonatal mice following staining with anti-PCNA antibody (green) and DAPI (blue). PCNA⁺ cells (green) were more abundantly present in HYP PL mice as compared to HYP AMD. Magnification×400. Scale Bar is 50 μm. FIG. 6B is a graph demonstrating significantly decreased number of PCNA⁺ cells/pulmonary vessel in the HYP AMD mice as compared to HYP PL, (* p<0.005 RA vs. Hyp PL; ** p<0.04 Hyp PL vs. Hyp AMD; n=4/group). FIG. 6C shows that phosphorylated-Akt expression was markedly decreased in the lungs of neonatal mice with PH following inhibition of the SDF-1/CXCR4 axis, (* p<0.0005 RA vs. Hyp PL; ** p<0.03 Hyp PL vs. Hyp AMD; n=4/group). FIG. 6D shows scans of photographs demonstrating that the Tunel Assay revealed significantly less apoptotic (pink) pulmonary vascular cells following inhibition of the SDF-1/CXCR4 axis. Magnification×400 . Scale Bar is 50 μm. FIG. 6E is a graph showing that there were significantly decreased number of Tunel⁺ cells/pulmonary vessel in the HYP AMD mice as compared to HYP PL, ((* p<0.02 RA vs. Hyp PL; ** p<0.05 Hyp PL vs. Hyp AMD; n=4/group).

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Exposure of the neonatal lung to chronic hypoxia produces significant pulmonary vascular remodeling, right ventricular hypertrophy (RVH) as well as decreased lung alveolarization. Based on the idea that stem cells contribute to pulmonary vascular remodeling and RVH, the hypothesis that blockade of stromal derived factor-1 (SDF-1), a key stem cell mobilizer or its receptor, chemokine receptor 4 (CXCR4) would attenuate and reverse hypoxia-induced cardiopulmonary remodeling, was tested. The experimental details are provided in the examples section which follows. The results showed that compared to the control, inhibition of the SDF-1/CXCR4 axis significantly improved lung alveolarization, as well as decreased pulmonary hypertension, RVH, vascular remodeling, vascular cell proliferation and lung or RV stem cell expressions to near baseline values. It was concluded that the SDF-1/CXCR4 axis both prevents and reverses hypoxia-induced cardiopulmonary remodeling in neonatal mice, by decreasing progenitor cell recruitment to the pulmonary vasculature as well as by decreasing pulmonary vascular cell proliferation. These data offer novel patho-physiologic insights into the role of the SDF-1/CXCR4 axis in the pathogenesis of neonatal hypoxia-induced cardiopulmonary remodeling and have important therapeutic implications.

### Definitions

The terms used herein have the meanings recognized and known to those of skill in the art, however, for convenience and completeness, particular terms and their meanings are set forth below. The terminology used herein is for the purpose of describing particular embodiments only and is not intended to be limiting of the invention.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Agent” refers to all materials that may be used to prepare pharmaceutical and diagnostic compositions, or that may be compounds such as small synthetic or naturally derived organic compounds, organic or inorganic compounds, synthetic or naturally derived, mimetics, carbohydrates, chimeric molecules (e.g. fusion proteins, spliced nucleic acids etc.) nucleic acids, polypeptides, antibodies, fragments, isoforms, variants, or other materials that may be used independently for such purposes, all in accordance with the present invention. The term “modulator” can be used interchangeably with “agent” when referring to anti-SDF-1 molecules or CXCR4 antagonists.

“Agonist” refers to an agent that mimics or up-regulates (e.g., potentiates or supplements) the bioactivity of a protein. An agonist may be a wild-type protein or derivative thereof having at least one bioactivity of the wild-type protein. An agonist may also be a compound that up-regulates expression of a gene or which increases at least one bioactivity of a protein. An agonist may also be a compound which increases the interaction of a polypeptide with another molecule, e.g., a target peptide or nucleic acid.

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate; ligand-receptor interactions etc. An antagonist may also be a compound that down-regulates expression of a gene or which reduces the amount of expressed protein present.

A “small molecule” refers to a composition that has a molecular weight of less than 3 kilodaltons (kDa), and preferably less than 1.5 kilodaltons, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, and preferably less than 1.5 kilodaltons, and more preferably less than about 1 kDa.

The concept of “combination therapy” is well exploited in current medical practice. Treatment of a pathology by combining two or more agents that target the same pathogen or biochemical pathway sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). As used herein, the term “combination therapy” means the two or more compounds can be delivered in a simultaneous manner, e.g. concurrently, or wherein one of the compounds is administered first, followed by the second agent, e.g. sequentially. The desired result can be either a subjective relief of one or more symptoms or an objectively identifiable improvement in the recipient of the dosage.

“Modulation” or “modulates” or “modulating” refers to up regulation (i.e., activation or stimulation), down regulation (i.e., inhibition or suppression) of a response, or the two in combination or apart.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted. In the present invention, the treatments using the agents described may be provided to treat patients suffering from a cardiac disorders such as for example, pulmonary hypertension due to stem cell mobilization as described in the examples section which follows.

“Subject” or “patient” refers to an animal or mammal, preferably a human, in need of treatment for a condition, disorder or disease.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Stem Cells” are cells, which are not terminally differentiated and are therefore able to produce cells of other types. Stem cells are divided into three types, including totipotent, pluripotent, and multipotent. “Totipotent stem cells” can grow and differentiate into any cell in the body, and thus can grow into an entire organism. These cells are not capable of self-renewal. In mammals, only the zygote and early embryonic cells are totipotent. “Pluripotent stem cells” are true stem cells, with the potential to make any differentiated cell in the body, but cannot contribute to making the extraembryonic membranes (which are derived from the trophoblast). “Multipotent stem cells” are clonal cells that self-renew as well as differentiate to regenerate adult tissues. “Multipotent stem cells” are also referred to as “unipotent” and can only become particular types of cells, such as blood cells or bone cells. The term “stem cells”, as used herein, refers to pluripotent stem cells capable of self-renewal.

A “niche” refers to a small zone within the microenvironment of a stem cell that maintains and controls stem cell activity in several organs.

“Adult stem cells” can be found in adult beings. Adult stem cells reproduce daily to provide certain specialized cells, for example 200 billion red blood cells are created each day in the body. Until recently it was thought that each of these cells could produce just one particular type of cell. This is called differentiation. However, in the past few years, evidence has been gathered of stem cells that can transform into several different forms. Bone marrow stem cells are known to be able to transform into liver, nerve, muscle and kidney cells. Stem cells isolated from the bone marrow have been found to be pluripotent. Useful sources of adult stem cells are found in organs throughout the body. In the same way that organs can be transplanted from cadavers, researchers have found that these could be used as a source of stem cells as well. Taking stem cells from the brains of corpses they were able to coax them into dividing into valuable neurons.

“Chemokines” (chemoattractant cytokines) are a family of homologous serum proteins of between 7 and 16 kDa, which were originally characterized by their ability to induce migration of leukocytes. Most chemokines have four characteristic cysteines (Cys), and depending on the motif displayed by the first two cysteines, they have been classified into CXC or alpha, CC or beta, C or gamma, and CX3C or delta chemokine classes. Two disulfide bonds are formed between the first and third cysteines and between the second and fourth cysteines. The only exception to the four cysteine motif is lymphotactin, which has only two cysteine residues. Thus, lymphotactin retains a functional structure with only one disulfide bond.

In addition, the CXC, or alpha, subfamily has been divided into two groups depending on the presence of the ELR motif (Glu-Leu-Arg) preceding the first cysteine: the ELR-CXC chemokines and the non-ELR-CXC chemokines (see, e.g., Belperio et al., “CXC Chemokines in Angiogenesis,” J. Leukoc. Biol. 68:1-8, 2000). ELR-CXC chemokines, such as IL-8, are generally strong neutrophil chemoattractants while non-ELR chemokines, such as IP-10, and SDF-1, predominantly recruit lymphocytes. CC chemokines, such as RANTES, MIP-1-alpha, MCP-1, generally function as chemoattractants for monocytes, basophils, eosinophils, and T-cells but not neutrophils. In general, chemokines are chemotactic agents that recruit leukocytes to the sites of injuries.

“CXCL12”, also known as stromal cell-derived factor-1 or “SDF-1” refers to a CXC chemokine that demonstrates in vitro activity with respect to lymphocytes and monocytes but not neutrophils. It is highly potent in vivo as a chemoattractant for mononuclear cells. SDF-1 has been shown to induce intracellular actin polymerization in lymphocytes, and to induce a transient elevation of cytoplasmic calcium in some cells. By “function of a chemokine, CXCL12” is meant the binding of the chemokine to its receptor and the subsequent effects on signaling. The nucleic acid sequence of the human CXCL12 is found in the following GenBank Accession numbers: NM 000609; NM001033886 BC039893; AY644456; AY802782 and CR450283.

When used in context of describing the invention, the terms “SDF-1” and “CXCR4” encompass all variants, alleles, analogs, isoforms, derivatives, species variants, fragments and the like.

“Chemokine Receptors” are G-protein coupled seven-transmembrane receptors. Based on the chemokine class they bind, the receptors have been named CXCR1, CXCR2, CXCR3, CXCR4, and CXCR5 (all of which bind CXC chemokines); CCR1 through CCR9 (all of which bind CC chemokines); XCR1 (which binds the C chemokine, Lptn); and CX3CR1 (which binds the CX3C chemokine, fractalkine or neurotactin). The CXCR4 receptor binds CXCL12. The nucleic acid sequence of human CXCR4 can be found in the following GenBank accession numbers: NM 001008540; Y14739; BC020968; AF052572; and AF025375.

“Analog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention, but need not necessarily comprise a compound that is similar or identical to those compounds of the preferred embodiment, or possess a structure that is similar or identical to the agents of the present invention.

“Derivative” refers to the chemical modification of molecules, either synthetic organic molecules or proteins, nucleic acids, or any class of small molecules such as fatty acids, or other small molecules that are prepared either synthetically or isolated from a natural source, such as a plant, that retain at least one function of the active parent molecule, but may be structurally different. Chemical modifications may include, for example, replacement of hydrogen by an alkyl, acyl, or amino group. It may also refer to chemically similar compounds which have been chemically altered to increase bioavailability, absorption, or to decrease toxicity. A derivative polypeptide is one modified by glycosylation, pegylation, or any similar process that retains at least one biological or immunological function of the polypeptide from which it was derived.

As used herein, the term “candidate compound” or “test compound” or “test agent” refers to any compound or molecule that is to be tested. As used herein, the terms, which are used interchangeably, refer to biological or chemical compounds such as simple or complex organic or inorganic molecules, peptides, proteins, oligonucleotides, polynucleotides, carbohydrates, or lipoproteins. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

RNA interference (RNAi) is an evolutionarily conserved mechanism in plant and animal cells that directs the degradation of messenger RNAs homologous to short double-stranded RNAs termed “small interfering RNA (siRNA)”. The ability of siRNA to direct gene silencing in mammalian cells has raised the possibility that siRNA might be used to investigate gene function in a high throughput fashion or to modulate gene expression in human diseases. Methods of preparing siRNAs are known to those skilled in the art. The following references are incorporated herein by reference in their entirety: Reich et al., Mol. Vis. 9:210-6 (2003); Gonzalez-Alegre P et al., Ann Neurol. 53:781-7 (2003); Miller et al., Proc Natl Acad Sci USA. (2003); Bidere et al., J Biol. Chem., published as manuscript M301911200 (Jun. 2, 2003); Van De Wetering et al., EMBO Rep. 4:609-15 (2003); Miller and Grollman, DNA Repair (Amst) 2:759-63 (2003); Kawakami et al., Nat Cell Biol. 5:513-9 (2003); Abdelrahim et al., Mol. Pharmacol. 63:1373-81 (2003); Williams et al., J Immunol. 170:5354-8 (2003); Daude et al., J Cell Sci. 116:2775-9 (2003); Jackson et al., Nat. Biotechnol. 21:635-7 (2003); Dillin, Proc Natl Acad Sci USA. 100:6289-91 (2003); Matta et al., Cancer Biol Ther. 2:206-10 (2003); Wohlbold et al., Blood. (2003); Julien and Herr, EMBO J. 22:2360-9 (2003); Scherr et al., Cell Cycle. 2:251-7 (2003); Giri et al., J Immunol. 170:5281-94 (2003); Liu and Erikson, Proc Natl Acad Sci USA. 100:5789-94 (2003); Chi et al., Proc Natl Acad Sci USA. 100:6343-6 (2003); Hall and Alexander, J Virol. 77:6066-9 (2003).

In the context of this invention, the term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), ed nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoögsteen types of base pairing, or the like.

As used herein, the term “animal” or “patient” is meant to include, for example, humans, sheep, elks, deer, mule deer, minks, mammals, monkeys, horses, cattle, pigs, goats, dogs, cats, rats, mice, birds, chicken, reptiles, fish, insects and arachnids.

“Mammal” covers warm blooded mammals that are typically under medical care (e.g., humans and domesticated animals). Examples include feline, canine, equine, bovine, and human, as well as just human.

### Prevention, Treatment and Compositions Modulating SDF-1/CXCR4

Pulmonary hypertension (PH) remains a significant cause of morbidity and mortality in humans and is especially severe in neonates who have been perinatally exposed to hypoxia. While the mechanisms of neonatal hypoxia-induced cardiopulmonary remodeling remain unclear, it may be that stem cells contribute to systemic as well as pulmonary vascular remodeling. The role of a key stem cell mobilizer, the chemokine stromal derived factor-1 (SDF-1) and its receptor chemokine receptor 4 (CXCR4) in neonatal chronic hypoxia-induced cardiopulmonary remodeling was examined.

SDF-1 or CXCL12 is a chemokine which is secreted by several tissues following exposure to hypoxia, in turn leading to the release of progenitor cells along a chemical gradient to the zone of tissue injury. Its receptor CXCR4, is a G-protein coupled receptor that is widely expressed on several tissues, including endothelial cells, smooth muscle cells, monocytes, hematopoietic and tissue committed stem cells. Binding of SDF-1 to CXCR4 induces several signal transduction pathways which regulate cell survival and proliferation.

Several therapies have been put forth for pulmonary hypertension, however most are directed at vasodilating the pulmonary circulation. The therapy described herein differs in many respects in that it also decreases pulmonary vascular remodeling, by directly targeting the cells which contribute to pulmonary vasculature thickening; decreases mobilization of stem cells to the pulmonary vasculature and may also decrease the associated cor pulmonale. Other conditions include for example: right heart failure, right ventricular failure, right ventricular hypertrophy, RVH, right ventricular dilatation, pulmonary hypertension, idiopathic primary pulmonary hypertension, cardiopulmonary disease, emphysema, pulmonary thromboembolism, interstitial lung disease, polycythemia vera, sickle cell disease, macroglobulinemia, chronic obstructive pulmonary disease, COPD, chronic bronchitis, pulmonary embolism, pulmonary emboli, exertional dyspnea, syncope with exertion, cor pulmonale, and the like.

In preferred embodiments, methods of preventing, reversing, or treating patients with cardiovascular conditions associated with hypoxia comprise administering a composition which modulates the SDF-1/CXCR4 axis in vivo and prevents the stem cells from migrating to cardiac tissues and/or retains stem cells in the bone marrow.

In another preferred embodiment, the methods of preventing, reversing, or treating patients with cardiovascular conditions associated with hypoxia comprise administering a composition which modulates the SDF-1/CXCR4 axis in vivo and includes an anti-inflammatory agent or cytokine.

In another preferred embodiment, a method of preventing or treating pulmonary hypertension comprises administering to a patient a therapeutically effective dose of a composition comprising at least one modulator of stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier.

In another preferred embodiment, a method of modulating SDF-1/CXCR4 signaling pathway in vitro or in vivo comprises contacting a cell, organ or tissue, or administering to a patient a therapeutically effective dose of at least one modulator of stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier.

In another preferred embodiment, a method of preventing, treating, or reversing hypoxia-induced pulmonary vascular remodeling in vivo comprising administering to a patient a therapeutically effective dose of at least one modulator of stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier.

Stem cells can be identified by various means, Preferably the stem cells are identified by stem cell markers. Examples of stem cell markers include, c-kit⁺, Sca-1⁺, IsI-1⁺ and the like.

In another preferred embodiment, a method of preventing, reversing, or treating disorders associated with hypoxia comprises administering to a patient a therapeutically effective dose of at least one modulator of stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier.

As discussed infra, prophylactic measures, treating or reversing a cardiac condition associated with hypoxia can include combination therapies. For example, two or more anti-SDF-1 agents, CXCR4 antagonists, and/or chemotherapeutic agents, such as for example, anti-inflammatory agents, agents which inhibit cytokines associated with the inflammation, adrenergic receptor agonists or antagonists, antisense oligonucleotides, anti-growth factor agents, anti-cytokine agents, and the like.

Compositions: In a preferred embodiment, a composition which is administered to a patient, modulates expression and/or function for the SDF-1/CXCR4 axis. The compositions comprise nucleic acids, oligonucleotides, polynucleotides, peptides, polypeptides, enzymes, small molecules, organic or inorganic molecules and the like. For example, CXCR4 can be modulated by an antagonist such as AMD3100 (1,1′-[1,4-phenylenebis(methylene)]bis-1,4,8,11-tetraazacyclotetradecane), or mimetics thereof. AMD3100 (also known as PLERIXAFOR, rINN, USAN, MOZOBIL, JM 3100) is a symmetric bicyclam, prototype non-peptide antagonist of the CXCR4 chemokine receptor. Mutational substitutions at 16 positions located in TM-III, -IV, -V, -VI, and -VII lining the main ligand-binding pocket of the CXCR4 receptor have identified three acid residues: Asp¹⁷¹ (AspIV:20), Asp²⁶² (AspVI:23), and Glu²⁸⁸ (GluVII:06) as the main interaction points for AMD3100. Molecular modeling suggests that one cyclam ring of AMD3100 interacts with Asp¹⁷¹ in TM-IV, whereas the other ring is sandwiched between the carboxylic acid groups of Asp²⁶² and Glu²⁸⁸ from TM-VI and -VII, respectively. In one study, it was found that introduction of only a Glu at position VII:06 and the removal of a neutralizing Lys residue at position VII:02 resulted in a 1000-fold increase in affinity of AMD3100 to within 10-fold of its affinity in CXCR4. Thus, mimetics, such as for example, peptide or non-peptide antagonists with improved oral bioavailability can be designed to efficiently and selectively block the CXCR4 receptor.

By function is meant the ability of the chemokine, e.g. SDF-1, to bind to its receptor and initiate the signaling cascade. In another embodiment, the CXCR4 antagonist is characterized by its ability to block or antagonize the expression and/or function of CXCR4. By function is meant the ability of the chemokine receptor to bind to its ligand or a mimic/mimetic thereof and initiate the signaling cascade.

In another preferred embodiment, a composition comprises a modulator that inhibits SDF-1 expression and/or function. For example, an anti-SDF-1 antibody binds to SDF-1 preventing engagement of its receptor. In another example, antisense oligonucleotides could decrease expression of SDF-1 polynucleotides in a cell, thereby decreasing SDF-1 proteins or peptides thereof.

In another preferred embodiment, a composition comprises a modulator that inhibits chemokine receptor 4 (CXCR4) expression and/or function. For example, antagonists such as for example, AMD3100, anti-CXCR4 antibodies, compounds which down regulate CXCR4 receptor expression, antisense oligonucleotides etc.

In another preferred embodiment, a composition comprises modulators of SDF-1 and CXCR4. For example, a composition can comprise an anti-SDF-1 antibody and a CXCR4 antagonist such as for example, AMD3100. Compounds which can modulate the SDF-1/CXCR4 axis comprise nucleic acids, oligonucleotides, polynucleotides, peptides, polypeptides, antibodies, aptamers, small molecules, organic molecules, inorganic molecules, synthetic or natural, or combinations thereof.

The combinations referred to above may conveniently be presented for use in the form of a pharmaceutical formulation and thus pharmaceutical formulations comprising a combination as defined above together with a pharmaceutically acceptable carrier thereof comprises a further aspect of the invention. The individual components of such combinations may be administered either sequentially or simultaneously in separate or combined pharmaceutical formulations.

Various agonists and antagonists of the natural ligands or receptors can be produced. Receptor binding assays can be developed. See, e.g., Bieri, et al. (1999) Nature Biotechnology 17:1105-1108. Calcium flux assays may be developed to screen for compounds possessing antagonist activity. Migration assays may take advantage of the movement of cells through pores in membranes, which can form the basis of antagonist assays. Chemotaxis may be measured thereby. Alternatively, chemokinetic assays may be developed, which measure the induction of kinetic movement, not necessarily relative to a gradient, per se. Chemokine agonists will exhibit some or all of the signaling functions of the chemokine, e.g., binding, inducing a Ca⁺⁺ flux, and chemoattracting appropriate receptor bearing cells. Conversely, antagonists will block the signaling and/or effector biology. Various mammalian chemokine sequences may be evaluated to determine what residues are conserved across species, suggesting what residues may be changed without dramatic effects on biological activity. Alternatively, conservative substitutions in certain regions of the molecule are somewhat more likely to maintain receptor binding activity, while other regions will more likely affect signal transduction. Standard methods for screening mutant or variant chemokine polypeptides will determine what sequences will be useful therapeutic antagonists.

In addition, certain nucleic acid expression methods may be applied. For example, in certain contexts, it may be useful to transfect cells with various nucleic acids which will be expressed, as appropriate. Various promoters may be operably linked to the gene, thereby allowing for regulated expression, e.g., suppression.

Antagonist activity may be tested or screened for using well known methods. Tests for ability to antagonize chemokine binding, calcium flux, or chemoattractant activity can be developed. Various ligand homologs can be created which retain receptor binding capacity, but lack signaling capability, thus serving as competitive binding molecules. Small molecules may also be screened for ability to antagonize chemokine function, e.g., chemoattraction, receptor binding, Ca⁺⁺ flux, and other effects mediated by chemokine. See generally Gilman, et al. (eds. 1990) Goodman and Gilman's: The Pharmacological Bases of Therapeutics, 8th Ed., Pergamon Press; Remington's Pharmaceutical Sciences, 17th ed. (1990), Mack Publishing Co., Easton, Pa., each of which is incorporated herein by reference. Agonists or antibodies may function as means to target, e.g., for labeling or specific localization.

CXCR4 antagonist compounds of the invention may include SDF-1 derivatives, such as C-terminal hydroxymethyl derivatives, O-modified derivatives (e.g., C-terminal hydroxymethyl benzyl ether), N-terminally modified derivatives including substituted amides such as alkylamides and hydrazides and compounds in which a C-terminal phenylalanine residue is replaced with a phenethylamide analogue (e.g., Ser-Ile-phenethylamide as an analogue of the tripeptide Ser-Ile-Phe). Within a CXCR4 antagonist compound, a peptidic structure (such as an SDF-1 derived peptide) maybe coupled directly or indirectly to at least one modifying group. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the SDF-1 core peptidic structure). For example, the modifying group can be coupled to the amino-terminus or carboxyterminus of an SDF-1 peptidic structure, or to a peptidic or peptidomimetic region flanking the core domain. Alternatively, the modifying group can be coupled to a side chain of at least one amino acid residue of a SDF-1 peptidic structure, or to a peptidic or peptido-mimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

In some embodiments, the modifying group may comprise a cyclic, heterocyclic or polycyclic group. The term “cyclic group”, as used herein, includes cyclic saturated or unsaturated (i.e., aromatic) group having from 3 to 10, 4 to 8, or 5 to 7 carbon atoms. Exemplary cyclic groups include cyclopropyl, cyclobutyl, cyclopentyl, cyclohexyl, and cyclooctyl. Cyclic groups may be unsubstituted or substituted at one or more ring positions. A cyclic group may for example be substituted with halogens, alkyls, cycloalkyls, alkenyls, alkynyls, aryls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN.

The term “heterocyclic group” includes cyclic saturated, unsaturated and aromatic groups having from 3 to 10, 4 to 8, or 5 to 7 carbon atoms, wherein the ring structure includes about one or more heteroatoms. Heterocyclic groups include pyrrolidine, oxolane, thiolane, imidazole, oxazole, piperidine, piperazine, morpholine. The heterocyclic ring may be substituted at one or more positions with such substituents as, for example, halogens, alkyls, cycloalkyls, alkenyls, alkynyls, aryls, other heterocycles, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN. Heterocycles may also be bridged or fused to other cyclic groups as described below.

The term “polycyclic group” as used herein is intended to refer to two or more saturated, unsaturated or aromatic cyclic rings in which two or more carbons are common to two adjoining rings, so that the rings are “fused rings”. Rings that are joined through non-adjacent atoms are termed “bridged rings. Each of the rings of the polycyclic group may be substituted with such substituents as described above, as for example, halogens, alkyls, cycloalkyls, alkenyls, alkynyls, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, or —CN.

The term “alkyl” refers to the radical of saturated aliphatic groups, including straight chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl has 20 or fewer carbon atoms in its backbone (C₁-C₂₀ for straight chain, C₃-C₂₀ for branched chain), or 10 or fewer carbon atom. In some embodiments, cycloalkyls may have from 4-10 carbon atoms in their ring structure, such as 5, 6 or 7 carbon rings. Unless the number of carbons is otherwise specified, “lower alkyl” as used herein means an alkyl group, as defined above, having from one to ten carbon atoms in its backbone structure. Likewise, “lower alkenyl and “lower alkynyl” have chain lengths of ten or less carbons.

The term “alkyl” (or “lower alkyl”) as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The terms “alkenyl” and “alkynyl” refer to unsaturated aliphatic groups analogous in length and possible substitution to the alkyls described above, but that contain at least one double or triple bond respectively.

The term “aralkyl”, as used herein, refers to an alkyl or alkylenyl group substituted with at least one aryl group. Exemplary aralkyls include benzyl (i.e., phenylmethyl), 2-naphthylethyl, 2-(2-pyridyl)propyl, 5-dibenzosuberyl, and the like.

The term “alkylcarbonyl”, as used herein, refers to —C(O)-alkyl. Similarly, the term “arylcarbonyl” refers to —C(O)-aryl. The term “alkyloxycarbonyl”, as used herein, refers to the group —C(O)—O-alkyl, and the term “aryloxycarbonyl” refers to —C(O)—O aryl. The term “acyloxy” refers to —O—C(O)—R₇, in which R₇ is alkyl, alkenyl, alkynyl, aryl, aralkyl or heterocyclyl.

The term “amino”, as used herein, refers to —N(R_{α )(R}_{β}), in which R_{α} and R_{β} are each independently hydrogen, alkyl, alkyenyl, alkynyl, aralkyl, aryl, or in which R_{α} and R_{β} together with the nitrogen atom to which they are attached form a ring having 4-8 atoms. Thus, the term “amino”, as used herein, includes unsubstituted, monosubstituted (e.g., monoalkylamino or monoarylamino), and disubstituted (e.g., dialkylamino or alkylarylamino) amino groups. The term “amido” refers to —C(O)—N(R₈)(R₉), in which R₈ and R₉ are as defined above. The term “acylamino” refers to —N(R′₈)C(O)—R₇, in which R₇ is as defined above and R′₈ is alkyl. As used herein, the term “nitro” means —NO₂; the term “halogen” designates —F, —Cl, —Br or —I; the term “sulfhydryl” means —SH; and the term “hydroxyl” means —OH.

The term “aryl” as used herein includes 5-, 6- and 7-membered aromatic groups that may include from zero to four heteroatoms in the ring, for example, phenyl, pyrrolyl, furyl, thiophenyl, imidazolyl, oxazole, thiazolyl, triazolyl, pyrazoyl, pyridyl, pyrazinyl, pyridazinyl and pyrimidinyl, and the like. Those aryl groups having heteroatoms in the ring structure may also be referred to as “aryl heterocycles” or “heteroaromatics”. The aromatic ring can be substituted at one or more ring positions with such substituents as described above, as for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, a heterocyclyl, an aromatic or heteroaromatic moiety, —CF₃, —CN, or the like. Aryl groups can also be part of a polycyclic group. For example, aryl groups include fused aromatic moieties such as naphthyl, anthracenyl, quinolyl, indolyl, and the like.

Modifying groups may include groups comprising biotinyl structures, fluorescein-containing groups, a diethylene-triaminepentaacetyl group, a (−)-menthoxyacetyl group, a N-acetylneuraminyl group, a cholyl structure or an iminiobiotinyl group. A CXCR4 antagonist compound may be modified at its carboxy terminus with a cholyl group according to methods known in the art (see e.g., Wess, G. et al. (1993) Tetrahedron Letters, 34:817-822; Wess, G. et al. (1992) Tetrahedron Letters 33:195-198; and Kramer, W. et al. (1992) J. Biol. Chem. 267:18598-18604). Cholyl derivatives and analogues may also be used as modifying groups. For example, a preferred cholyl derivative is Aic (3-(O-aminoethyl-iso)-cholyl), which has a free amino group that can be used to further modify the CXCR4 antagonist compound. A modifying group may be a “biotinyl structure”, which includes biotinyl groups and analogues and derivatives thereof (such as a 2-iminobiotinyl group). In another embodiment, the modifying group may comprise a “fluorescein-containing group”, such as a group derived from reacting an SDF-1 derived peptidic structure with 5-(and 6-)-carboxyfluorescein, succinimidyl ester or fluorescein isothiocyanate. In various other embodiments, the modifying group(s) may comprise; an N-acetylneuraminyl group, a trans-4-cotininecarboxyl group, a 2-imino-1-imidazolidineacetyl group, an (S)-(−)-indoline-2-carboxyl group, a (−)-menthoxyacetyl group, a 2-norbornaneacetyl group, a oxo-5-acenaphthenebutyryl, a (−)-2-oxo-4-thiazolidinecarboxyl group, a tetrahydro-3-furoyl group, a 2-iminobiotinyl group, a diethylenetriaminepentaacetyl group, a 4-morpholinecarbonyl group, a 2-thiopheneacetyl group or a 2-thiophenesulfonyl group.

A CXCR4 antagonist compound of the invention may be further modified to alter the specific properties of the compound. For example, in one embodiment, the compound may be modified to alter a pharmacokinetic property of the compound, such as in vivo stability, bioavailability or half-life. The compound may be modified to label the compound with a detectable substance. The compound may be modified to couple the compound to an additional therapeutic moiety. To further chemically modify the compound, such as to alter its pharmacokinetic properties, reactive groups can be derivatized. For example, when the modifying group is attached to the amino-terminal end of the SDF-1 core domain, the carboxy-terminal end of the compound may be further modified. Potential C-terminal modifications include those that reduce the ability of the compound to act as a substrate for carboxypeptidases. Examples of C-terminal modifiers include an amide group, an ethylamide group and various non-natural amino acids, such as D-amino acids and β-alanine. Alternatively, when the modifying group is attached to the carboxy-terminal end of the aggregation core domain, the amino-terminal end of the compound may be further modified, for example, to reduce the ability of the compound to act as a substrate for aminopeptidases.

A CXCR4 antagonist compound can be further modified to label the compound by reacting the compound with a detectable substance. Suitable detectable substances include various enzymes, prosthetic groups, fluorescent materials, luminescent materials and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ⁹⁹mTc, ³⁵S or ³H. A CXCR4 antagonist compound may be radioactively labeled with ¹⁴C, either by incorporation of ¹⁴C into the modifying group or one or more amino acid structures in the CXCR4 antagonist compound. Labeled CXCR4 antagonist compounds may be used to assess the in vivo pharmacokinetics of the compounds, as well as to detect disease progression or propensity of a subject to develop a disease, for example for diagnostic purposes. Tissue distribution CXCR4 receptors can be detected using a labeled CXCR4 antagonist compound either in vivo or in an in vitro sample derived from a subject. For use as an in vivo diagnostic agent, a CXCR4 antagonist compound of the invention may be labeled with radioactive technetium or iodine. A modifying group can be chosen that provides a site at which a chelation group for the label can be introduced, such as the Aic derivative of cholic acid, which has a free amino group. For example, a phenylalanine residue within the SDF-1 sequence (such as amino acid residue 13) may be substituted with radioactive iodotyrosyl. Any of the various isotopes of radioactive iodine may be incorporated to create a diagnostic agent. ¹²³I (half-life=13.2 hours) may be used for whole body scintigraphy, ¹²⁴I (half life=4 days) may be used for positron emission tomography (PET), ¹²⁵I (half life=60 days) may be used for metabolic turnover studies and ¹³¹I (half life=8 days) may be used for whole body counting and delayed low resolution imaging studies.

In an alternative chemical modification, a CXCR4 antagonist compound may be prepared in a “prodrug” form, wherein the compound itself does not act as a CXCR4 antagonist, but rather is capable of being transformed, upon metabolism in vivo, into a CXCR4 antagonist compound as defined herein. For example, in this type of compound, the modifying group can be present in a prodrug form that is capable of being converted upon metabolism into the form of an active CXCR4 antagonist. Such a prodrug form of a modifying group is referred to herein as a “secondary modifying group.” A variety of strategies are known in the art for preparing peptide prodrugs that limit metabolism in order to optimize delivery of the active form of the peptide-based drug (see e.g., Moss, J. (1995) in Peptide-Based Drug Design: Controlling Transport and Metabolism, Taylor, M. D. and Amidon, G. L. (eds), Chapter 18.

CXCR4 antagonist compounds may be prepared by standard techniques known in the art. A peptide component of a CXCR4 antagonist may be composed, at least in part, of a peptide synthesized using standard techniques (such as those described in Bodansky, M. Principles of Peptide Synthesis, Springer Verlag, Berlin (1993); Grant, G. A. (ed.). Synthetic Peptides: A User's Guide, W. H. Freeman and Company, New York (1992); or Clark-Lewis, I., Dewald, B., Loetscher, M., Moser, B., and Baggiolini, M., (1994) J. Biol. Chem., 269, 16075-16081). Automated peptide synthesizers are commercially available (e.g., Advanced ChemTech Model 396; Milligen/Biosearch 9600). Peptides may be assayed for CXCR4 antagonist activity in accordance with standard methods. Peptides may be purified by HPLC and analyzed by mass spectrometry. Peptides may be dimerized via a disulfide bridge formed by gentle oxidation of the cysteines using 10% DMSO in water. Following HPLC purification dimer formation may be verified, by mass spectrometry. One or more modifying groups may be attached to a SDF-1 derived peptidic component by standard methods, for example using methods for reaction through an amino group (e.g., the alpha-amino group at the amino-terminus of a peptide), a carboxyl group (e.g., at the carboxy terminus of a peptide), a hydroxyl group (e.g., on a tyrosine, serine or threonine residue) or other suitable reactive group on an amino acid side chain (see e.g., Greene, T. W. and Wuts, P. G. M. Protective Groups in Organic Synthesis, John Wiley and Sons, Inc., New York (1991)).

In another aspect of the invention, CXCR4 antagonist peptides may be prepared according to standard recombinant DNA techniques using a nucleic acid molecule encoding the peptide. A nucleotide sequence encoding the peptide may be determined using the genetic code and an oligonucleotide molecule having this nucleotide sequence may be synthesized by standard DNA synthesis methods (e.g., using an automated DNA synthesizer). Alternatively, a DNA molecule encoding a peptide compound may be derived from the natural precursor protein gene or cDNA (e.g., using the polymerase chain reaction (PCR) and/or restriction enzyme digestion) according to standard molecular biology techniques.

In some embodiments, the CXCR4 antagonists for use in the invention may be substantially purified peptide fragments, modified peptide fragments, analogues or pharmacologically acceptable salts of either SDF-1α or SDF-1β. SDF-1 derived peptide antagonists of CXCR4 may be identified by known physiological assays and a variety of synthetic techniques (such as disclosed in Crump et al., 1997, The EMBO Journal 16(23) 6996-7007; and Heveker et al., 1998, Current Biology 8(7): 369-376; each of which are incorporated herein by reference). Such SDF-1 derived peptides may include homologs of native SDF-1, such as naturally occurring isoforms or genetic variants, or polypeptides having substantial sequence similarity to SDF-1, such as 20%, 30%, 40%, 50%, 60%, 70%, 80%, 90%, 95% or 99% sequence identity to at least a portion of the native SDF-1 sequence, the portion of native SDF-1 being any contiguous sequence of 10, 20, 30, 40, 50 or more amino acids, provided the peptides have CXCR4 antagonist activity. In some embodiments, chemically similar amino acids may be substituted for amino acids in the native SDF-1 sequence (to provide conservative amino acid substitutions).

In another preferred embodiment, a composition comprises a combination of two or more modulators of SDF-1 expression and/or function, modulators of CXCR4 expression and/or function, and chemotherapeutic agents. The combinations referred to above may conveniently be presented for use in the form of a pharmaceutical formulation and thus pharmaceutical formulations comprising a combination as defined above together with a pharmaceutically acceptable carrier thereof comprises a further aspect of the invention.

The individual components of such combinations may be administered either sequentially or simultaneously in separate or combined pharmaceutical formulations.

Treatment of PAH includes lifestyle modifications, conventional treatments, and disease-specific treatments. Examples of other chemotherapeutics for use in combination therapies include statins, elastase inhibitors, nitrix oxides, phosphodiesterase inhibitors, L-arginine, antiplatelet agents, serotonin inhibitors, agents to alter ion channel function, gene therapy, vasoactive intestinal peptide (VIP), antiproliferative heparins, tyrosine kinase inhibitors, calcium channel blockers, prostacyclins e.g. epoprostenol, treprostinil, endothelin receptor antagonists e.g. bosentan, and the like.

The HMG-CoA reductase inhibitor statins confer potent anti-proliferative and anti-inflammatory cardiovascular benefit, in addition to cholesterol-lowering effects. Statins suppress endothelial and vascular smooth muscle cell neointimal responses to vascular injury in animal models.

In rats subjected to hypoxia or monocrotaline, serine elastase increases in the pulmonary arteries before vascular remodeling, related to phosphorylation of MAP kinase and induction of AML1-transactivating activity. Inhibition of elastase attenuates pulmonary hypertension and structural changes.

The SDF-1/CXCR4 modulators can be combined with other current medical therapy which includes supportive treatment, e.g., digitalis, diuretics, and supplemental oxygen; anticoagulation (warfarin); calcium channel blockade (in the minority of patients with sustained vasodilation); chronic intravenous epoprostenol; newer PGI2 formulations (intravenous or inhaled iloprost, subcutaneous, aerosol, and intravenous treprostinil, or oral beraprost); and endothelin receptor antagonists (Newman J. H. et al., Circulation, 2004; 109:2947-2952).

Combination therapies which include the anti-SDF-1 agents and CXCR4 antagonists with one or more chemotherapeutic agents are not limited to the above chemotherapeutic agents, but may include any alternatives that the physician may deem appropriate. The combination therapies also include combinations of two or more anti-SDF-1 agents, or CXCR4 antagonists, and/or anti-SDF-1 and CXCR4 antagonists. Examples of CXCR4 antagonists include, AMD3465 which is a monocyclam CXCR4 inhibitor; non-cyclam AMD1 1070; T134 which is a small analog (14 amino acid residues) of T22 (R. Arakaki et al., Journal of Virology, February 1999, p. 1719-1723, Vol. 73, No. 2); MSX-122;RCP168 (Z. Zeng et al. Mol Cancer Ther Dec. 1, 2006 5, 3113). AMD1 1070 is the first orally bioavailable small-molecule CXCR4 inhibitor.

Antisense Therapy: The relationship between an antisense compound such as an oligonucleotide and its complementary nucleic acid target, to which it hybridizes, is commonly referred to as “antisense”. “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state. In the present invention, the targets are nucleic acids encoding CXCL12 or CXCR4; in other words, a gene encoding either CXCL12 or CXCR4, or mRNA expressed from the CXCL12 or CXCR4 gene. mRNA which encodes CXCL12 or CXCR4 is presently the preferred target. The targeting process also includes determination of a site or sites within the nucleic acid sequence for the antisense interaction to occur such that modulation of gene expression will result.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention, which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding CXCL12 or CXCR4, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region,” “AUG region” and “translation initiation codon region” refer to a portion of such an mRNA or gene that encompasses from about 25 to about 50 contiguous nucleotides in either direction (i.e., 5′ or 3′) from a translation initiation codon. This region is a particular target region. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is a particular target region. The open reading frame (ORF) or “coding region,” which is known in the art to refer to the region between the translation initiation codon and the translation termination codon, is also a region which may be targeted effectively. Other preferred target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

The overall effect of interference with mRNA function is modulation of expression of CXCL12 or CXCR4. In the context of this invention “modulation” means either inhibition or stimulation; i.e., either a decrease or increase in expression. This modulation can be measured in ways which are routine in the art, for example by Northern blot assay of mRNA expression, or reverse transcriptase PCR, or by Western blot or ELISA assay of protein expression, or by an immunoprecipitation assay of protein expression. Effects on cell proliferation can also be measured. Inhibition is presently preferred.

Pharmaceutical compositions comprising the oligonucleotides of the present invention (any antisense oligonucleotides or siRNA molecules) may include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33). One or more penetration enhancers from one or more of these broad categories may be included. Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems 1991, page 92; Muranishi, Critical Reviews in Therapeutic Drug Carrier Systems 1990, 7, 1; E1-Hariri et al., J. Pharm. Pharmacol. 1992 44, 651-654).

Screening Methods for Identifying Agents that Decrease the Expression or Function of CXCL12 or that Antagonize CXCR4.

The present invention further provides for a method of discovery of agents or compounds which modulate mobilization of stem cells or progenitor cells. Thus, in a preferred embodiment, a method of identifying candidate agents which modulate stromal derived factor-1 (SDF-1) and/or chemokine receptor 4 (CXCR4) expression and/or function comprises obtaining a stromal derived factor-1 (SDF-1) molecule, chemokine receptor 4 (CXCR4) molecule or cell expressing at least one of these molecules; contacting said molecules or cell expressing at least one of these molecules with a candidate agent; measuring expression and/or function of SDF-1 and CXCR4 as compared to a normal control.

Experimental details are provided in the examples section which follows in identifying candidate therapeutic agents which inhibit the SDF-1/CXCR4 axis. Other methods that may be utilized to determine whether a molecule functions to decrease the expression of CXCL12 or to act as a CXCR4 antagonists include, but are not limited to, the following: Inhibition of the induction of CXCL12 (SDF-1) receptor mediated rise in free cytosolic Ca²⁺ concentration ([Ca²⁺]) in response to native CXCL12 (or agonist analogs of CXCL12) (Loetscher P. et al., (1998) J. Biol. Chem. 273, 24966-24970), inhibition of SDF-1-induction of phosphoinositide-3 kinase or Protein Kinase C activity (Wang, J-F et al., (2000) Blood 95, 2505-2513), inhibition of SDF-1-induced migration of CD34⁺ hematopoietic stem cells in a two-chamber migration (transwell) assay (Durig J. et al., (2000) Leukemia 14, 1652-1660; Peled A. et al., (2000) Blood 95, 3289-2396), inhibition of SDF-1 associated transmigration of CD34⁺/CXCR4⁺ cells through vascular endothelial cells in a cell chemotaxis assay, cell adhesion assay, or real-time tracking of CD34⁺ cell migration in 3-D extracellular matrix-like gel assays (Peled A. et al., (2000) Blood 95, 3289-2396), inhibition of SDF-1 associated chemotaxis of marrow-derived B cell precursors (Nuzzo M. et al., Eur. J. Immunol. (1997) 27, 1788-1793), preventing CXCR4 signal transduction and coreceptor function in mediating the entry of T- and dual-tropic HIV isolates (Zhou N. et al., (2000) 39, 3782-3787), inhibition of SDF-1 associated increases of CFU-GM, CGU-M or BFU-E colony formation by peripheral blood Inc⁺ CD34⁺ progenitor cells (Lataillade J-J. et all. (2000) Blood 95, 756-768), or inhibition of integrin-mediated adhesion of T cells to fibronectin and ICAM-1 (Buckley C. D et al., (2000) J. Immunology 165, 3423-3429). Where it is necessary to assess the inhibition of CXCL12 associated mechanisms in the aforementioned assays, various concentrations of CXCR4 antagonist may be incubated under the appropriate experimental conditions in the presence of CXCL12, in assays to determine if the CXCR4 antagonist associated repression of the respective mechanism results directly from inhibition of the CXCR4 receptor. ([Ca²⁺]) mobilization, chemotaxis assays or other assays that measure the induction of CXCR4 are not limited to the cell types indicated in the associated references, but may include other cell types that demonstrate CXCR4 associated, and specific, activation.

The number and identity of stem cells can be quantified based on a variety of methods, see, for example, the examples section which follows. The number of stem cells may be quantitated by methods, including fluorescent activated cell sorting, whereby the cells are labeled with particular markers specific for hematopoietic stem cells or progenitor cells. For example, cells having the following phenotype are indicative of the presence of hematopoietic stem cells: lin⁻ sca⁻¹ c-kit⁺. Alternatively, undifferentiated hematopoietic stem cells or progenitor cells from the bone marrow, when cultured in methyl cellulose with stromal cells, will migrate under the stromal layer and demonstrate a very characteristic cobblestone appearance. Upon addition of an adrenergic agonist or a test agent that acts to mobilize the hematopoietic stem cells or progenitor cells, the undifferentiated stem cells will migrate from under the stromal cells into the supernatant. The number of these cells in the supernatant can then be counted and surface markers identified using standard procedures known to those skilled in the art, for example, by flow cytometric procedures.

In alternative aspects, the invention provides uses for CXCR4 antagonists that are identified as molecules that bind to CXCR4 (whether reversible or irreversible) and are associated with the repression of CXCR4 associated activity. Binding affinity of a CXCR4 antagonists may for example be associated with ligand binding assay dissociation constants (K_{D}) in the range of a minimum of 1 pM, 10 pM, 100 pM, 1 μM, 10 μM or 100 μM up to a maximum of 1 mM, or any value in any such range. CXCR4 antagonist associated KD values may be determined through alternative approaches, such as standard methods of radioligand binding assays, including High Throughput Fluorescence Polarization, scintillation proximity assays (SPA), and FLASHPLATES™ (Allen et al., (2000) J. Biomolecular Screening 5, 63-69), where the competing ligand is native SDF-1. Alternatively, the affinity of a CXCR4 antagonist for the SDF-1 receptor (CXCR4) may be ascertained through inhibition of native SDF-1 binding to the CXCR4, where various concentrations of the CXCR4 antagonist are added in the presence of SDF-1 and a recombinant CXCR4 or a cell type that expresses an adequate receptor titer.

Binding to or interaction with a CXCR4 receptor may be determined by performing an assay such as, e.g., a binding assay between a desired compound and a CXCR4 receptor. In one aspect, this is done by contacting said compound to a CXCR4 receptor and determining its dissociation rate. Numerous possibilities for performing binding assays are well known in the art. The indication of a compound's ability to bind to the receptor is determined, e.g., by a dissociation rate, and the correlation of binding activity and dissociation rates is well established in the art. For example, the assay may be performed by radio-labeling a reference compound, or other suitable radioactive marker, and incubating it with the cell bearing a CXCR4 receptor. Test compounds are then added to these reactions in increasing concentrations. After optimal incubation, the reference compound and receptor complexes are separated, e.g., with chromatography columns, and evaluated for bound ¹²⁵I-labeled peptide with a gamma (γ) counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{reference}). A small RIC⁻¹ value indicates strong relative binding, whereas a large RIC⁻¹ value indicates weak relative binding. See, for example, Latek et al., Proc. Nat. Acad. Sci. USA, Vol. 97, No. 21, pp. 11460-11465, 2000. A receptor agonist or antagonist mimic may be computationally evaluated and designed by means of a series of steps in which chemical groups or fragments are screened and selected for their ability to associate with the individual binding pockets or interface surfaces of the protein (e.g. the receptor). One skilled in the art may employ one of several methods to screen chemical groups or fragments for their ability to associate with the receptor. This process may begin by visual inspection of, for example, the protein/protein interfaces or the binding site on a computer screen based on the available crystal complex coordinates of the receptor, including a protein known to interact with the receptor. Selected fragments or chemical groups may then be positioned in a variety of orientations, or docked, at an individual surface of the receptor that participates in a protein/protein interface or in the binding pocket. Docking may be accomplished using software such as QUANTA and SYBYL, followed by energy minimization and molecular dynamics with standard molecular mechanics forcefields, such as CHARMM and AMBER (AMBER, version 4.0 (Kollman, University of California at San Francisco); QUANTA/CHARMM (Molecular Simulations, Inc., Burlington, Mass). Specialized computer programs may also assist in the process of selecting fragments or chemical groups. These include: GRID (Goodford, 1985, J. Med. Chem. 28:849-857), available from Oxford University, Oxford, UK; MCSS (Miranker & Karplus, 1991, Proteins: Structure, Function and Genetics 11:29-34), available from Molecular Simulations, Burlington, Mass.; AUTODOCK (Goodsell & Olsen, 1990, Proteins: Structure, Function, and Genetics 8:195-202), available from Scripps Research Institute, La Jolla, Calif.; and DOCK (Kuntz et al., 1982, J. Mol. Biol. 161:269-288), available from University of California, San Francisco, Calif. Once suitable chemical groups or fragments that bind to the receptor have been selected, they can be assembled into a single compound. Assembly may proceed by visual inspection of the relationship of the fragments to each other in the three-dimensional image displayed on a computer screen in relation to the structure coordinates thereof. This would be followed by manual model building using software such as QUANTA or SYBYL. Useful programs to aid one of skill in the art in connecting the individual chemical groups or fragments include: CAVEAT (Bartlett et al., 1989, ‘CAVEAT: A Program to Facilitate the Structure-Derived Design of Biologically Active Molecules.’ In Molecular Recognition in Chemical and Biological Problems', Special Pub., Royal Chem. Soc. 78:182-196), available from the University of California, Berkeley, Calif.; 3D Database systems such as MACCS-3D (MDL Information Systems, San Leandro, Calif.). This area is reviewed in Martin, 1992, J. Med. Chem. 35:2145-2154); and HOOK (available from Molecular Simulations, Burlington, Mass.). Instead of proceeding to build a receptor agonist or antagonist mimic, in a step-wise fashion one fragment or chemical group at a time, as described above, such compounds may be designed as a whole or de novo using either an empty binding site or the surface of a protein that participates in protein/protein interactions or optionally including some portion(s) of a known activator(s). These methods include: LUDI (Bohm, 1992, J. Comp. Aid. Molec. Design 6:61-78), available from Molecular Simulations, Inc., San Diego, Calif.; LEGEND (Nishibata & Itai, 1991, Tetrahedron 47:8985), available from Molecular Simulations, Burlington, Mass.; and LeapFrog (available from Tripos, Inc., St. Louis, Mo.). Other molecular modeling techniques may also be employed in accordance with this invention. See, e.g., Cohen et al., 1990, J. Med. Chem. 33:883-894. See also, Navia & Murcko, 1992, Current Opinions in Structural Biology 2:202-210.

Once a compound has been designed by the above methods, the efficiency with which that compound may bind to or interact with the receptor protein may be tested and optimized by computational evaluation. Agonists or antagonists may interact with the receptor in more than one conformation that is similar in overall binding energy. In those cases, the deformation energy of binding is taken to be the difference between the energy of the free compound and the average energy of the conformations observed when the inhibitor binds to the receptor protein.

A compound selected for binding to the receptors may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the inhibitor and the receptor protein when the mimic is bound to it preferably make a neutral or favorable contribution to the enthalpy of binding. Specific computer software is available in the art to evaluate compound deformation energy and electrostatic interaction. Examples of programs designed for such uses include: Gaussian 92, revision C (Frisch, Gaussian, Inc., Pittsburgh, Pa); AMBER, version 4.0 (Kollman, University of California at San Francisco); QUANTA/CHARMM (Molecular Simulations, Inc., Burlington, Mass.); and Insight II/Discover (Biosym Technologies Inc., San Diego, Calif.). These programs may be implemented, for instance, using a computer workstation, as are well-known in the art. Other hardware systems and software packages will be known to those skilled in the art.

Once a receptor modulating compound has been optimally designed, for example as described above, substitutions may then be made in some of its atoms or chemical groups in order to improve or modify its binding properties, or its pharmaceutical properties such as stability or toxicity. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to the adrenergic receptor by the same computer methods described in detail above.

Candidate Compounds and Agents: Examples of agents, candidate compounds or test compounds include, but are not limited to, nucleic acids (e.g., DNA and RNA), carbohydrates, lipids, proteins, peptides, peptidomimetics, small molecules, aptamers, and other drugs. In one preferred aspect, agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Phage display libraries may be used to screen potential ligands or receptor modulators. Their usefulness lies in the ability to screen, for example, a library displaying a billion different compounds with only a modest investment of time, money, and resources. For use of phage display libraries in a screening process, see, for instance, Kay et al., Methods, 240-246, 2001. An exemplary scheme for using phage display libraries to identify compounds that are agonists of the adrenergic receptor or that act as mobilizers of stem cells may be described as follows: initially, an aliquot of the library is introduced into microtiter plate wells that have previously been coated with target protein, e.g. an adrenergic receptor. After incubation (e.g. 2 hrs), the nonbinding phage are washed away, and the bound phage are recovered by denaturing or destroying the target with exposure to harsh conditions such as, for instance pH 2, but leaving the phage intact. After transferring the phage to another tube, the conditions are neutralized, followed by infection of bacteria with the phage and production of more phage particles. The amplified phage are then rescreened to complete one cycle of affinity selection. After three or more rounds of screening, the phage are plated out such that there are individual plaques that can be further analyzed. For example, the conformation of binding activity of affinity-purified phage for an adrenergic receptor may be obtained by performing ELISAs. One skilled in the art can easily perform these experiments. In one aspect, a receptor molecule used for any of the assays may be selected from a recombinant adrenergic receptor protein, or a fusion protein, an analog, derivative, or mimic thereof.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869 or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

The methods of screening compounds may also include the specific identification or characterization of such compounds, whose stem cell mobilization was determined by the methods described herein. If the identity of the compound is known from the start of the experiment, no additional assays are needed to determine its identity. However, if the screening for compounds that modulate the receptor is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan (see for instance neogenesis.com). Neogenesis' ALIS (automated ligand identification system) spectral search engine and data analysis software allow for a highly specific identification of a ligand structure based on the exact mass of the ligand. One skilled in the art can also readily perform mass spectrometry experiments to determine the identity of the compound.

Antibodies, including polyclonal and monoclonal antibodies, particularly anti-CXCL12 or anti-CXCR4 antibodies may be useful as compounds to modulate stem cell mobilization and can be used in conjunction with another chemotherapeutic agent. CXCL12 or CXCR4 may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of CXCL12 or CXCR4 may be discovered or synthesized, and may be used in diagnostic and/or therapeutic protocols.

### Pharmaceutical Formulations

As explained above, the present methods can, for example, be carried out using a single pharmaceutical composition comprising both a CXCR4 and SDF-1 antagonist when administration is to be simultaneous or sequential.

Pharmaceutical compositions employed in the methods of the invention include a compound (e.g., a CXCR4 antagonist, anti-SDF-1 molecule, SDF-1 antagonist) formulated with other ingredients, e.g., “pharmaceutically acceptable carriers”. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Other pharmaceutical carriers include, but are not limited to, antioxidants, preservatives, dyes, tablet-coating compositions, plasticizers, inert carriers, excipients, polymers, coating materials, osmotic barriers, devices and agents which slow or retard solubility, etc. Non-toxic pharmaceutically acceptable excipients that are suitable for the manufacture of tablets include, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Anti-SDF-1 and/or CXCR4 antagonists (nucleic acids, polypeptides) preferably are produced recombinantly, although such molecules may be isolated from biological extracts. Alternatively, direct administration of cells encoding anti-SDF-1 and/or CXCR4 antagonists is also contemplated.

Recombinantly produced agents such as SDF-1 or anti-SDF-1 polypeptides, include chimeric proteins comprising a fusion of a SDF-1. protein with another polypeptide, e.g., a polypeptide capable of providing or enhancing protein-protein binding, sequence specific nucleic acid binding (such as GAL4), decreasing stability of the SDF-1 polypeptide under assay conditions, or providing a detectable moiety, such as green fluorescent protein. A polypeptide fused to a SDF-1 polypeptide or fragment which may also provide means of readily detecting the fusion protein, e.g., by immunological recognition or by fluorescent labeling.

Various techniques may be employed for introducing nucleic acids e.g. CXCR4 and/or SDF-1 sense and anti-sense, dominant negative) into cells, depending on whether the nucleic acids are introduced in vitro or in vivo in a host. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an aptamer, or antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Other delivery systems can include time-release, delayed release or sustained release delivery systems. Such systems can avoid repeated administrations of the agents, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

A delivery system of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV) which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., (1981) 6:77). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells: (3) delivery of the aqueous contents of the vesicle to the target cell cytoplasm at high efficiency: and (4) accurate and effective expression of genetic information.

Liposomes may be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one embodiment, the vehicle is a biocompatible microparticle or implant that is suitable for implantation into the mammalian recipient. Exemplary bioerodible implants that are useful in accordance with this method are described in PCT International application No. PCT/US/03307 (Publication No. WO 95/24929, entitled “Polymeric Gene Delivery System”). PCT/US/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the agents described herein are encapsulated or dispersed within the biocompatible, preferably biodegradable polymeric matrix disclosed in PCT/US/03307.

The polymeric matrix preferably is in the form of a microparticle such as a microsphere (wherein an anti-SDF-1 agent, or CXCR4 antagonist, for example, is dispersed throughout a solid polymeric matrix) or a microcapsule comprising an anti-SDF-1 agent, or CXCR4 antagonist, for example. Other forms of the polymeric matrix for containing an anti-SDF-1 agent, or CXCR4 antagonist include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used. Preferably when an aerosol route is used the polymeric matrix and fugetactic agent are encompassed in a surfactant vehicle. The polymeric matrix composition can be selected to have both favorable degradation rates and also to be formed of a material which is bioadhesive, to further increase the effectiveness of transfer. The matrix composition also can be selected not to degrade, but rather, to release by diffusion over an extended period of time.

In another important embodiment the delivery system is a biocompatible microsphere that is suitable for local, site-specific delivery. Such microspheres are disclosed in Chickering et al, Biotech. And Bioeng., (1996) 52:96-101 and Mathiowitz et al., Nature, (1997) 386:410-414.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is particularly desirable. The polymer optionally is in the form of a hydrogel that can absorb up to about 90% of its weight in water and further, optionally is cross-linked with multivalent ions or other polymers.

In general, an anti-SDF-1 agent, or CXCR4 antagonist can also be delivered using a bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules. (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In addition, important embodiments of the invention include pump-based hardware delivery systems, some of which are adapted for implantation. Such implantable pumps include controlled-release microchips. An example of a controlled-release microchip is described in Santini, J T Jr. et al., Nature, 1999, 397:335-338, the contents of which are expressly incorporated herein by reference.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In certain embodiments, the agents of the invention are delivered directly to the site via intra-cardial injection. In this manner, the compositions can be targeted locally to particular sites to modulate stem cell migration to these sites. In another example the local administration involves an implantable pump to the site in need of such treatment.

In another preferred embodiment of the invention, the agents of the invention are administered to a subject in combination with a balloon angioplasty procedure. A balloon angioplasty procedure involves inserting a catheter having a deflated balloon into an artery. The agent may be attached to the balloon angioplasty catheter in accordance with standard procedures known in the art. For example, the anti-SDF-1 molecule may be stored in a compartment of the balloon angioplasty catheter until the balloon is inflated, at which point it is released into the local environment. Alternatively, the molecule may be impregnated on the balloon surface, such that it contacts the cells of the arterial wall as the balloon is inflated. The molecule also may be delivered in a perforated balloon catheter such as those disclosed in Flugelman, et al., Circulation, v. 85, p, 1110-1117 (1992). See, also, e.g., published PCT Patent Application WO 95/23161, for an exemplary procedure for attaching a therapeutic protein to a balloon angioplasty catheter. This procedure can be modified using no more that routine experimentation to attach a therapeutic nucleic acid to the balloon angioplasty catheter.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Methods and Materials

Animal Care and Treatment: Sixty Eight FVB/NJ neonatal mice exposed to normobaric hypoxia (10% O₂) or normoxia (20.9% O₂) for one week were randomly assigned to receive daily intraperitoneal injections of normal saline (PL; n=19), or AMD3100 (7.5 mg/kg; n=10) or anti-SDF-1 antibody (25pg/kg; n=19) for seven days, from postnatal day 1-7.

Therapeutic Strategy: Twenty one FVB/NJ neonatal mice (1-2 d old) were exposed to normobaric hypoxia (10% O₂) or normoxia (20.9% O₂) for two weeks. After one week of this exposure, the mice were randomly assigned to receive daily intra-peritoneal injections of normal saline (PL; n=6), or AMD3100 (7.5 mg/kg; n=7) for seven days, from postnatal day 7-14. The experimental protocol was performed according to guidelines set forth by the University of Miami Animal Care and Use Committee.

### Example 1

### Inhibition of the SDF-1/CXCR4 Axis Attenuates Neonatal Hypoxia-Induced Pulmonary Hypertension

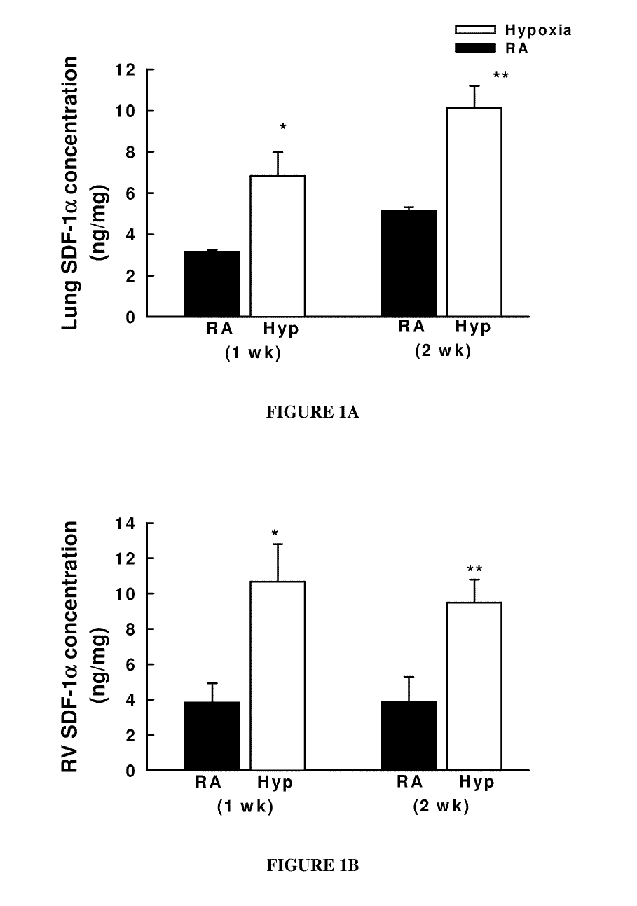

SDF-1 Expression in Neonatal Pulmonary Hypertension: Without wishing to be bound by theory, it was hypothesized that neonatal hypoxia up regulates pulmonary SDF-1. Indeed, after hypoxia for one or two wk, lung protein expression of SDF-1 increased 2 fold (FIG. 1A). Similarly, within the right ventricle SDF-1 was also markedly elevated (FIG. 1B). In contrast, left ventricle SDF-1 was not elevated in response to hypoxia.

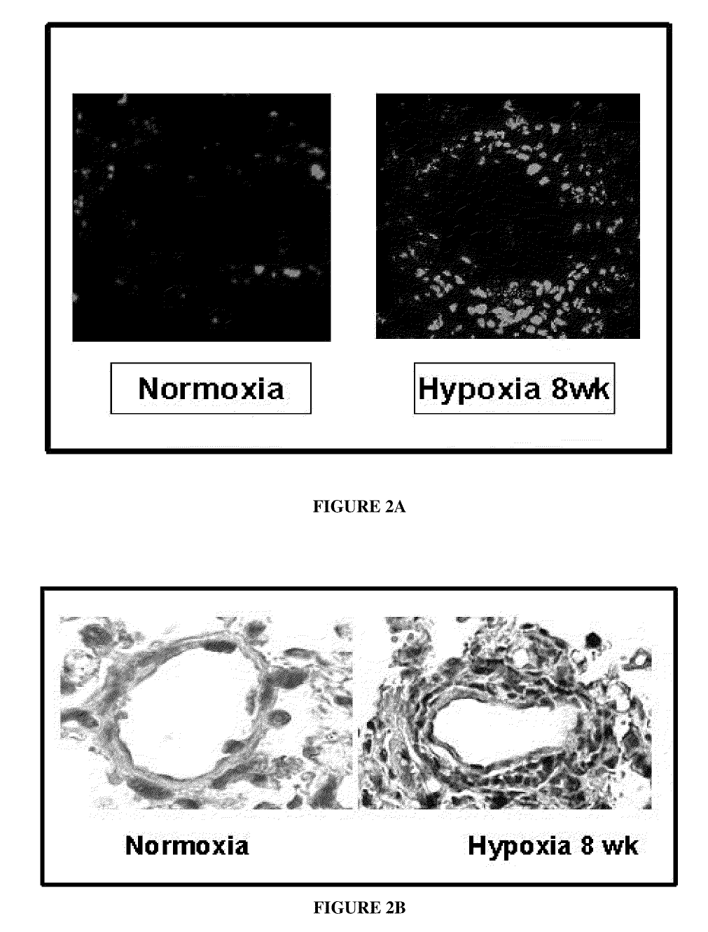

Bone Marrow -Derived Cells Migrate to the Pulmonary Vasculature during Hypoxia: It was next examined whether bone marrow- derived cells were recruited to the pulmonary vasculature during chronic hypoxia. Six week old mice whose bone marrow had been reconstituted with GFP+ cells were exposed to hypoxia for 8 wk. Following this exposure, GFP⁺ cells were visualized in the smooth muscle and adventitial layers of the hypoxic pulmonary arteries (FIGS. 2A and 2B).

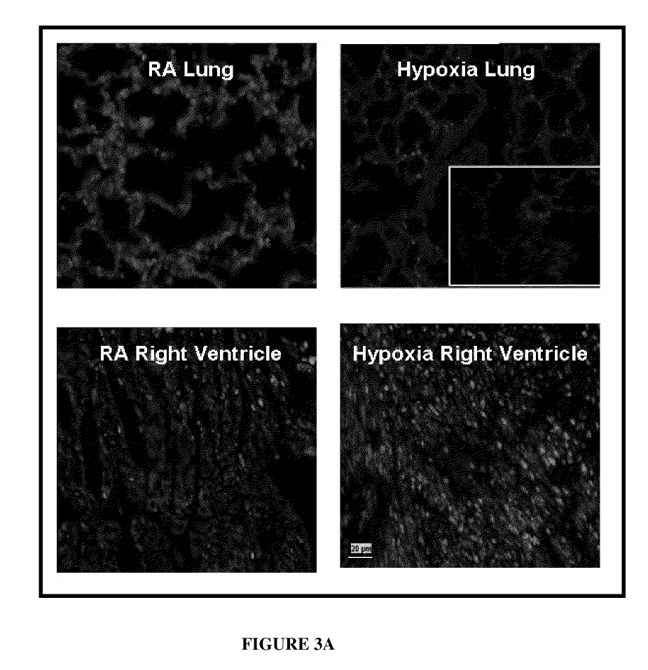

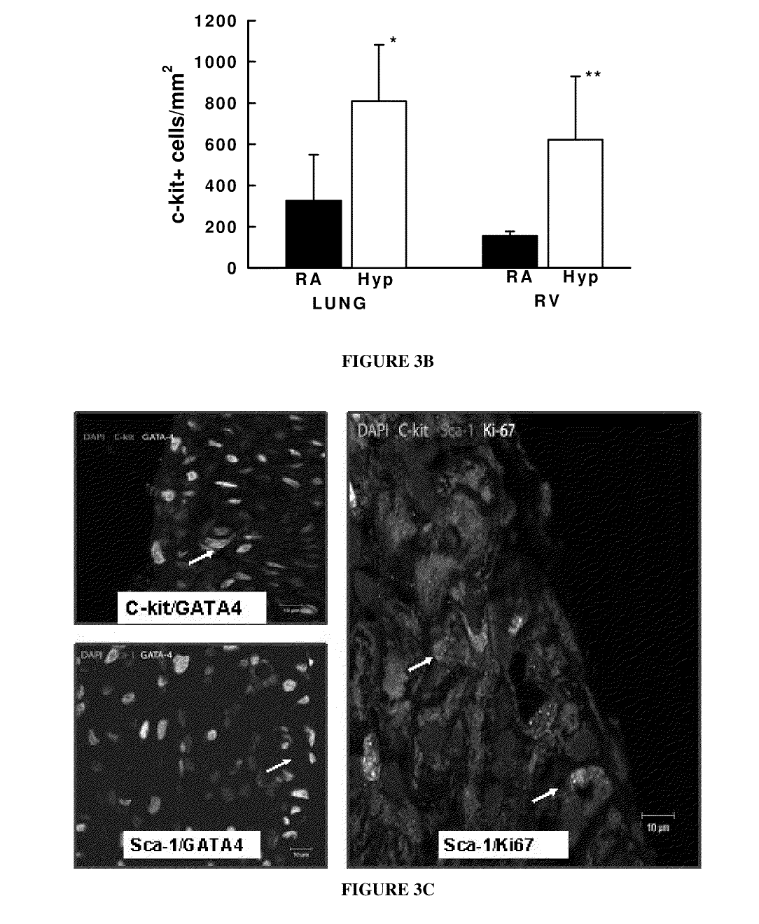

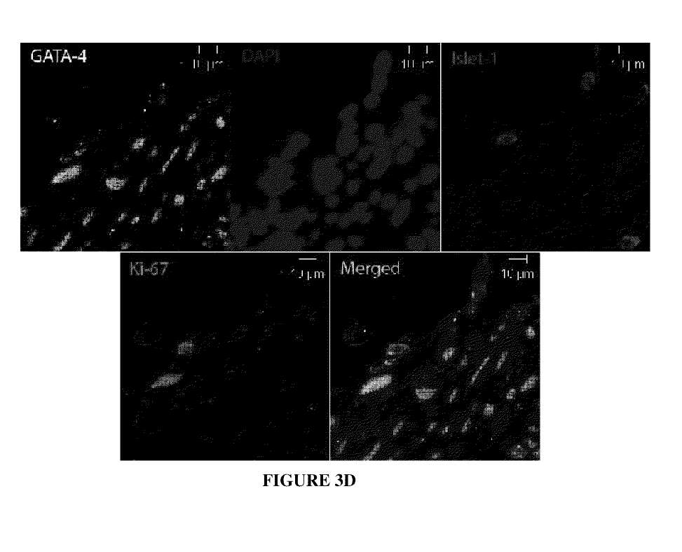

Given this, it was sought to determine whether neonatal mice (1-2 d) exposed to hypoxia had increased numbers of c-kit+cells in their lungs and right ventricles. Indeed, a 2.5 and 4 fold increase in the number of c-kit⁺ cells in the lungs and right ventricles respectively of neonatal mice with PH and RVH (FIGS. 3A-3B) was demonstrated. These c-kit⁺ cells were localized mainly to the adventitia of the hypoxic pulmonary arterioles. Additionally, within the hypertrophied right ventricles, double Immunofluorescence study also demonstrated c-kit⁺, Sca-1⁺ and Isl-1⁺ cells co-localized with GATA-4 and Ki-67 suggesting that they were proliferating and committed to a cardiac fate (FIGS. 3C and 3D).

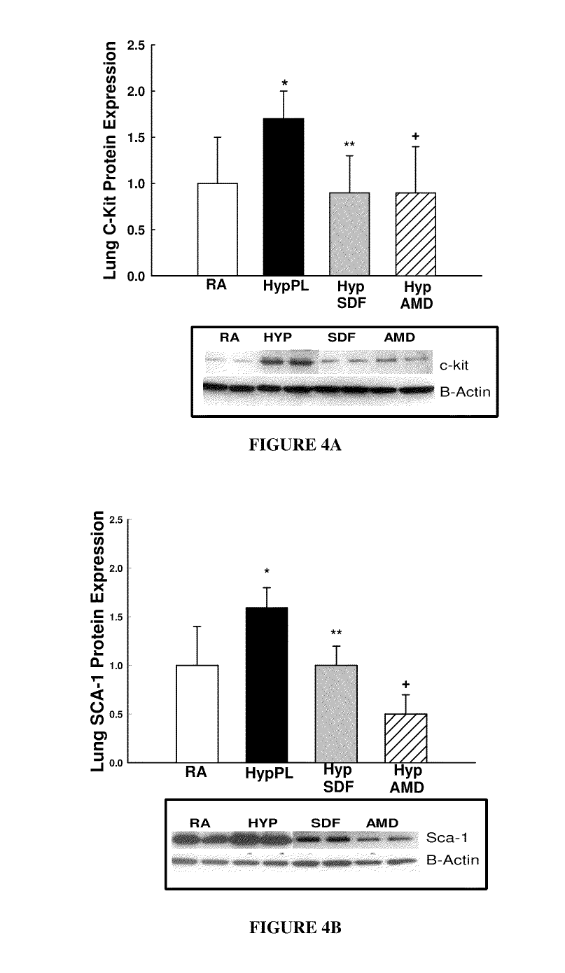

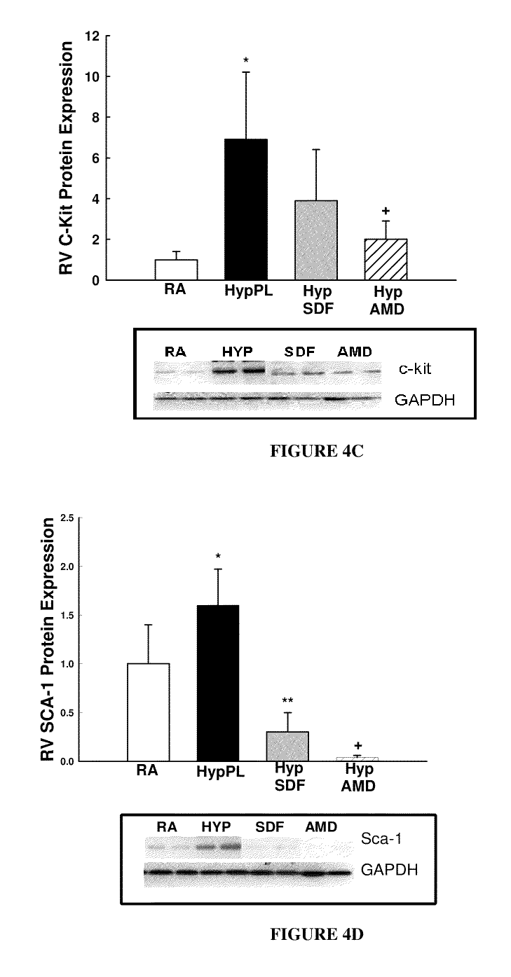

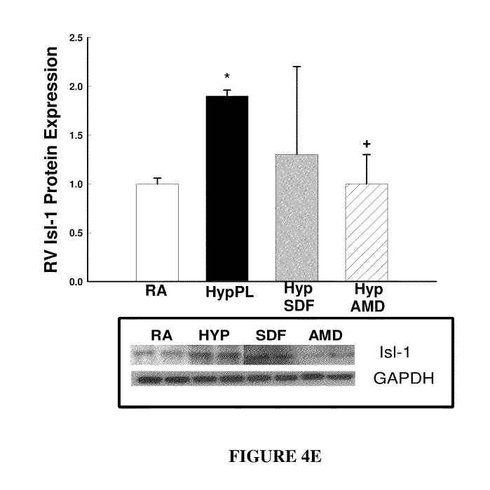

Inhibition of SDF-1/CXCR4 Axis Decreases Progenitor Cells in the Lungs and Right Ventricles of Mice with Pulmonary Hypertension: SDF-1 is a component of stem cell mobilization during hypoxia, and that c-kit⁺ as well as Sca-1⁺ cells were increased in the lungs and right ventricles of neonatal mice with hypoxia-induced PH, it was next evaluated whether inhibition of the SDF-1/CXCR4 axis would decrease the expression of c-kit and Sca-1 in the lungs and right ventricles of mice with PH. Administration of AMD3100 or anti-SDF-1 antibody to mice exposed to 1 wk hypoxia significantly decreased the protein expression of these stem cell markers to baseline or near baseline values (FIGS. 4A-4E).

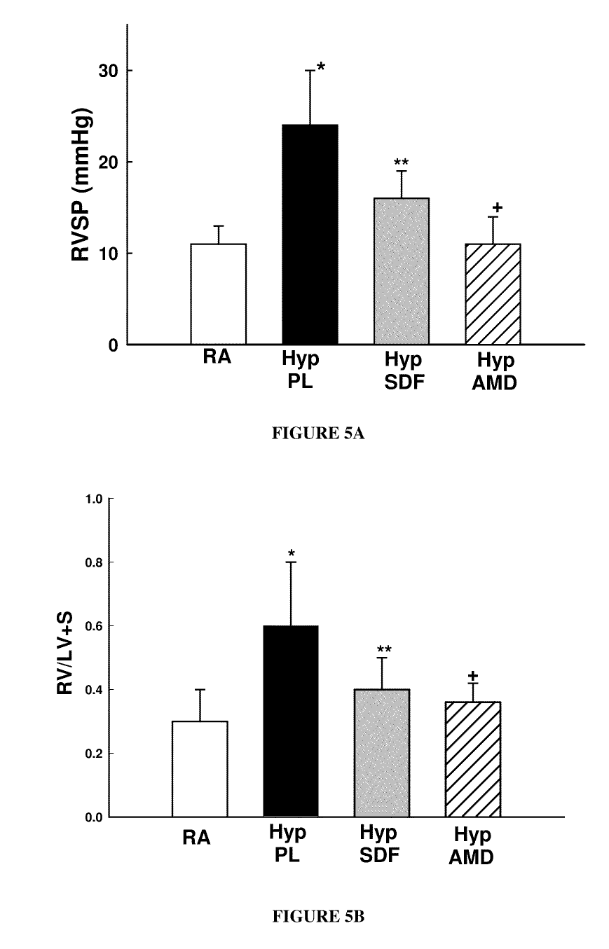

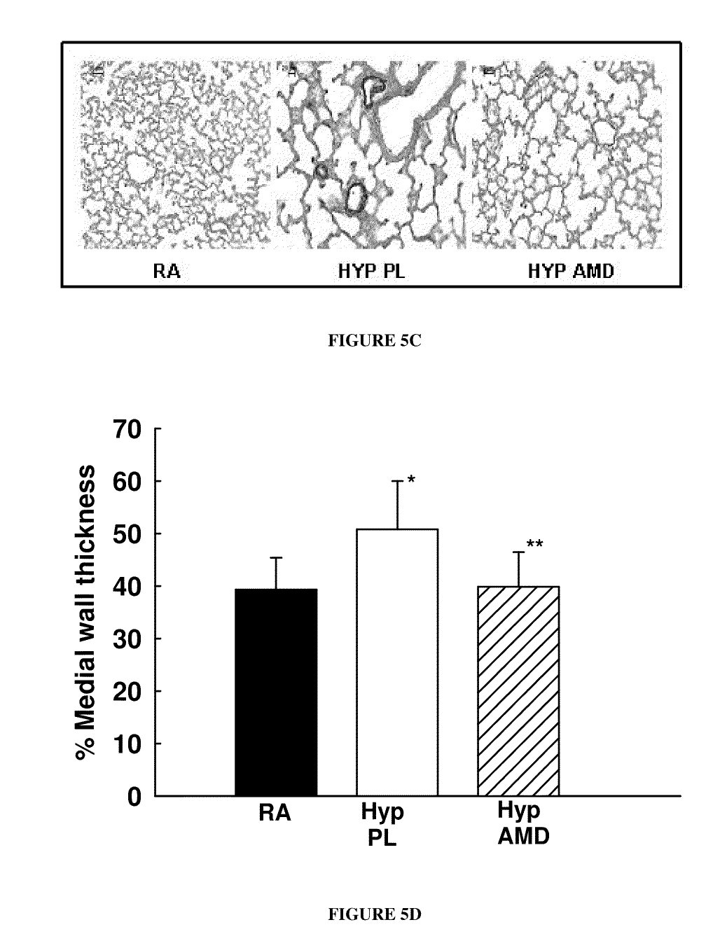

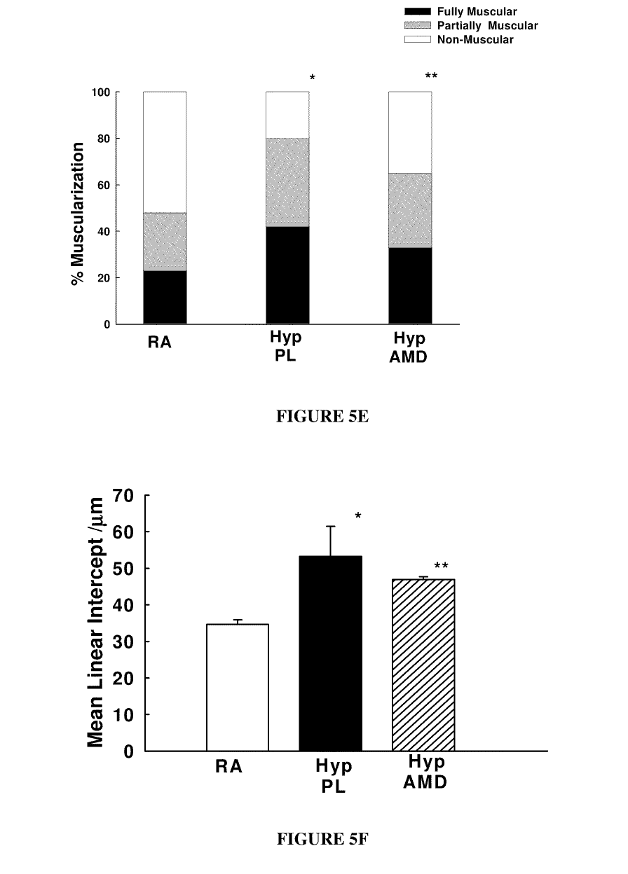

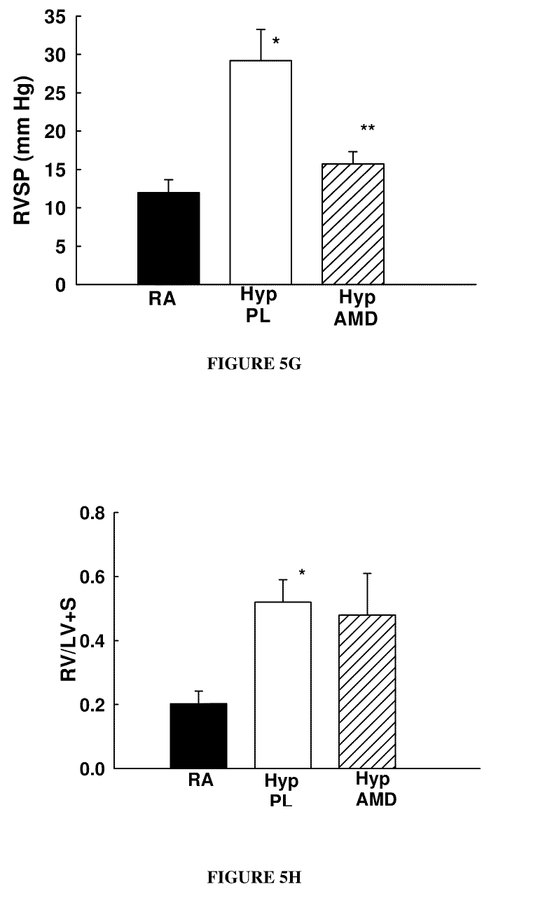

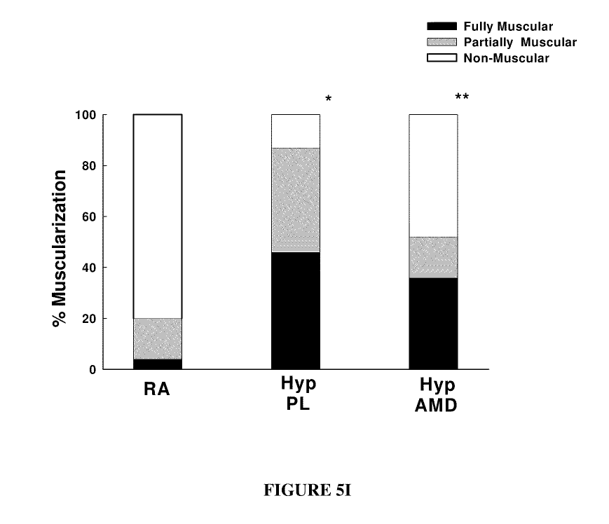

Inhibition of the SDF-1/CXCR4 axis Prevents and Reverses Pulmonary Vascular Remodeling: Next, it was investigated whether inhibition of the SDF-1/CXCR4 axis would prevent or reverse pulmonary vascular remodeling. Exposure of neonatal mice to 1 wk hypoxia resulted in a significant increase in RVSP (11±2 vs 24±6 mmHg; RA vs Hyp PL, p<0.001) and RV/LV+S (0.2±0.1 vs 0.5±0.2; RA vs Hyp PL, p<0.01). To test the idea that the increased release of SDF-1 in the lungs of hypoxic neonatal mice exacerbates PH, monoclonal anti-SDF-1 antibody or AMD3100 (a CXCR4 antagonist) was administered daily to neonatal mice exposed to 1 wk hypoxia. Importantly, both strategies to inhibit the SDF-1/CXCR4 axis restored RVSP and RV/LV+S close to baseline values (FIGS. 5A and 5B). In addition, as compared to hypoxic placebo mice, hypoxic AMD3100 treated mice had a marked decrease in the medial wall thickness, and a con-comitant increased percentage of non-muscular vessels (FIGS. 5C-5E).

Moreover, since chronic hypoxia significantly alters lung alveolarization in the neonate, the mean linear intercept (MLI) was measured to evaluate the effect of the SDF-1/CXCR4 axis on lung alveolarization. Whilst exposure to chronic hypoxia was associated with a significant increase in the MLI, blockade of the SDF-1/CXCR4 axis significantly decreased the MLI (FIG. 5F).

Therapeutic Strategy: Exposure of neonatal mice to 2 wk hypoxia also resulted in a marked increase in RVSP (12±2 vs 29±4 mmHg; RA vs Hyp PL, p<0.001) and RV hypertrophy ((0.2±0.0 vs 0.5±0.1; RA vs Hyp PL, p<0.01). In contrast, administration of the CXCR4 antagonist, AMD3100, in mice with established PH resulted in a significant decrease in RVSP but no significant difference in RV hypertrophy (FIGS. 5G-5H). There was however a significant increase in the percentage of non-muscularized pulmonary arterioles in the hypoxia treated mice as compared to placebo (FIG. 5I).

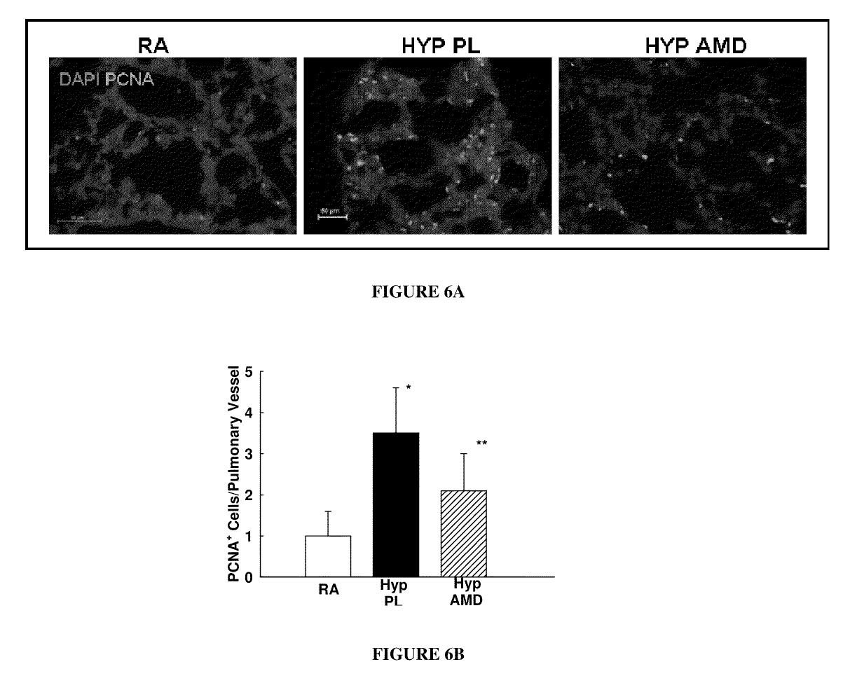

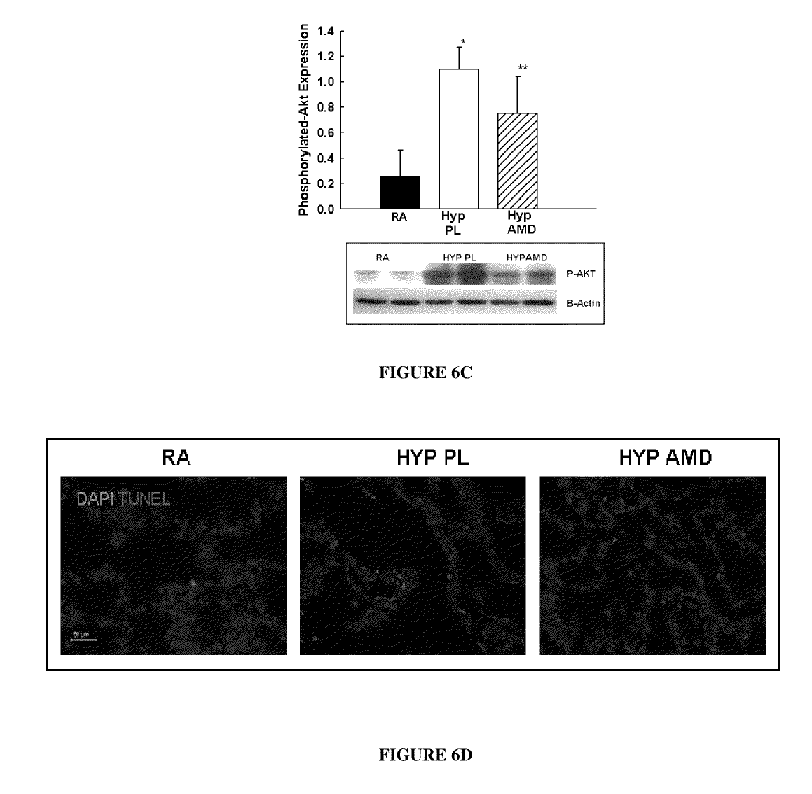

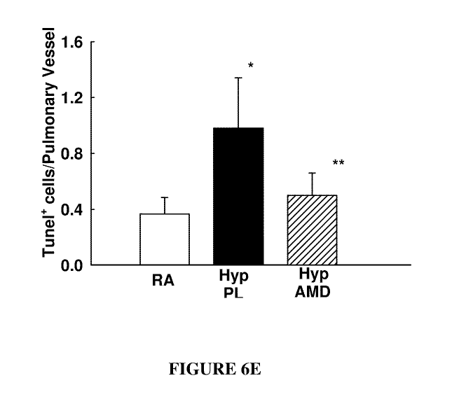

Inhibition of the SDF-1/CXCR4 Axis Decreases Pulmonary Vascular Cell Proliferation and Apoptosis: In order to ascertain other mechanisms by which inhibition of the SDF-1/CXCR4 axis attenuates hypoxia-induced pulmonary vascular remodeling, PCNA immunostaining and TUNEL assay were utilized to determine cell proliferation and survival respectively. Whilst exposure to hypoxia resulted in a marked increase in the number of PCNA⁺ and TUNEL⁺ cells in the pulmonary vasculature, this was significantly attenuated in the AMD3100 hypoxic mouse pups (FIGS. 6A-6E). Moreover, on further questioning of the down-stream signaling mechanisms that were responsible for this effect, a marked decrease in phosphorylated-Akt expression was demonstrated in the treated mouse pups as compared to placebo (FIG. 6C).

Discussion: The major new findings of this study are that direct systematic evidence of the participation was provided, as well as the mechanism of action of SDF-1 and its receptor CXCR4 in the pathogenesis of neonatal chronic hypoxia-induced cardiopulmonary remodeling. We demonstrate that inhibition of the SDF-1/CXCR4 axis improves alveolarization, prevents the development of hypoxia-induced pulmonary vascular remodeling in neonatal mice, and significantly decreases pulmonary artery pressure in neonatal mice with established disease. These findings were associated with decreased expression of progenitor cells in the lungs and right ventricles of treated mice as well as decreased pulmonary vascular cell proliferation and apoptosis. This study therefore offers important patho-physiologic insights into the role of the SDF-1/CXCR4 axis in neonatal chronic hypoxia-induced cardiopulmonary remodeling and has important therapeutic implications for neonatal hypoxia-induced PH.

One of the major roles of SDF-1 is the mobilization of stem cells from the bone marrow to injured sites 17-20. In proof of this concept, it was demonstrated that inhibition of the SDF-1/CXCR4 axis decreased the pulmonary expression of c-kit and sca-1, known stem cell markers. The role of c-kit⁺or sca-1⁺ cells in neonatal hypoxia-induced cardiopulmonary remodeling is however unclear. An increased number of c-kit cells in the pulmonary artery adventitia of neonatal animals with hypoxic PH 15 was demonstrated, whilst BM-derived cells express smooth muscle actin in hypoxia remodeled pulmonary arteries and selective depletion of circulating mesenchymal precursors prevented pulmonary adventitial remodeling (Frid M G, et al., Am J Pathol. 2006;168(2):659-669). The present study showed, among other things, inhibition of the SDF-1/CXCR4 axis both inhibited and reversed pulmonary vascular remodeling, and this was associated with decreased stem cell expression. This is plausible as SDF-1 may play a role in adult systemic vascular remodeling. In agreement with this, Satoh et al demonstrated increased expression of SDF-1 in the plasma of hypoxic adult rats and following administration of a statin, was able to show decreased pulmonary vascular remodeling associated with decrease SDF-1 and progenitor cell expression 26. Our study extends this finding and more importantly provides direct evidence that inhibition of the SDF-1/CXCR4 axis actually does decrease neonatal pulmonary vascular remodeling.

Another possible mechanism by which the SDF-1/CXC4 axis could participate in hypoxia-induced pulmonary vascular remodeling is through its role in cell proliferation and apoptosis. Vascular cell proliferation is an important component of pulmonary vascular remodeling, whilst early PH is associated with increased endothelial cell apoptosis and loss of small capillaries. It was demonstrated in this present study that inhibition of the SDF-1/CXCR4 axis decreased pulmonary vascular cell proliferation as well as apoptosis, and this was associated with decreased expression of phosphorylated Akt. Binding of SDF-1 to its receptor CXCR4 induces several signal transcription pathways including activation of phosphoinositol 3-kinase (PI3K). The PI3K/Akt axis affects the calcium currents that govern smooth muscle cell contraction through coupling membrane receptors to calcium channels, and SDF-1 may be a major regulator of smooth muscle cell proliferation through its involvement in P13K/PTEN signaling pathway. The data herein also further indicates that blockade of this SDF-1/CXCR4 pathway is not only therapeutically beneficial in PH through its effects on cell migration but also via its role in cell proliferation and survival.

Another important aspect of this study is the finding that inhibition of the SDF-1/CXCR4 axis also decreased right ventricular hypertrophy and this was associated with increased right ventricular expression of c-kit, Isl-1 and Sca-1. Moreover, cells which expressed these stem cell markers co-localized with GATA-4 and Ki67 evidencing that they were committed to a cardiac fate and also proliferating. It was also hypothesized as to whether these stem cells were bone marrow derived or resident stem cells. It was speculated that a significant fraction were indeed derived from the bone marrow, since following inhibition of the SDF-1/CXCR4 axis it was demonstrated that there was a significant decrease in the expression of these stem cell markers in the right ventricle. Nonetheless, the possible role of resident cardiac stem cells cannot be understated as our group have documented several resident stem niches within the myocardium which differentiate and proliferate in response to injury.

It is unclear whether these stem cells in the hypertrophied right ventricle have adaptive or maladaptive roles. Without wishing to be bound by theory, it was hypothesized that these cells do contribute significantly to the compensatory increase in myocardial mass that is necessary to adapt to the increase overload, and these findings extend to a role of stem cells in pressure- overload induced ventricular hypertrophy. A model of left ventricular overload induced by aortic stenosis showed some new formation of myocytes resulting from the differentiation of stem-like cells, stem cells or precursor cells could regenerate cardiomyocytes in hearts subjected to pressure overload, however, the roles of these stem cells may not be equivalent. This is the first study to demonstrate that Isl-1⁺ cells, contribute to right ventricular hypertrophy. This was a surprising finding as these cells were previously shown to be extremely sparse in the native heart after postnatal day 5 (Moretti A, et al., Cell. 2006;127(6):1151-1165; Laugwitz K L, et al., Nature. 2005;433(7026):647-653). Without wishing to be bound by theory, it was postulated that this response could be developmentally determined as our pilot studies evaluating the role of stem cells in pressure overload-induced right ventricular hypertrophy showed a progressive decline in the role of these cells with increasing age. It should also be noted that whilst these cells were significantly increased in the hypertrophied right ventricle, they were not changed in the left ventricle, indicating that the right ventricular stem cell response was not only due to global hypoxia of the animal, but rather a direct reflection of the increase in the SDF-1 expression noted in the right but not the left ventricle.

This finding is clearly surprising as hypoxia up regulates SDF-1 expression in several tissues in a manner proportional to the degree of hypoxia (Ceradini DJ, et al. Nat Med. 2004;10(8):858-864; Schioppa T, et al., J Exp Med. 2003;198(9):1391-1402). Additionally, within the promoter region of the SDF-1 gene there are at least two binding sites for the master regulator of hypoxic cell signaling, HIF-1α, and the latter is a major participant in hypoxia-induced cardiopulmonary remodeling (Ceradini D J, et al. Nat Med. 2004, 10(8):858-864; Yu A Y, et al., J Clin Invest. 1999;103(5):691-696). This present study does evidence that factors that are uniquely released in the pressure overloaded right ventricle may also regulate SDF-1 production. Indeed, chronic hypobaric hypoxia induces a differential transcriptional profile in the right and left ventricle, including genes involved in apoptosis, and SDF-1 secretion was triggered in medial smooth muscle cells following exposure to apoptotic bodies.

In conclusion, this study clearly demonstrates that SDF-1 participates in the pathogenesis of neonatal hypoxia-induced cardiopulmonary remodeling through several mechanisms. Without wishing to be bound by theory, it is proposed that during neonatal hypoxia, there is increased release of SDF-1 which results in the mobilization of progenitor cells to the pulmonary vasculature and right ventricle, and these mobilized cells may directly participate in pulmonary vascular remodeling and RVH. Moreover, following binding of the locally released SDF-1 to its receptor CXCR4, downstream signaling pathways regulate pulmonary vascular cell proliferation and apoptosis resulting in the findings evidenced in the lungs and hearts of hypoxic neonates. Finally, the demonstration that inhibition of SDF-1/CXCR4 axis significantly improves alveolarization, attenuates neonatal pulmonary hypertension, vascular remodeling and right ventricular hypertrophy indicates a novel and highly effective therapeutic strategy for this disease.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A composition comprising at least one modulator of stromal derived factor-1 (SDF-1), chemokine receptor 4 (CXCR4), a stromal derived factor-1 (SDF-1) and chemokine receptor 4 (CXCR4), or combinations thereof, in a therapeutically effective dose in a pharmaceutical carrier.

2. The composition of claim 1, wherein the at least one modulator of stromal derived factor-1 (SDF-1) comprising: nucleic acids, oligonucleotides, polynucleotides, peptides, polypeptides, antibodies, aptamers, small molecules, organic molecules, inorganic molecules, or combinations thereof.

3. The composition of claim 1, wherein the at least one modulator of chemokine receptor 4 (CXCR4) comprising: nucleic acids, oligonucleotides, polynucleotides, peptides, polypeptides, antibodies, aptamers, small molecules, organic molecules, inorganic molecules, or combinations thereof.

4. The composition of claim 1, wherein the at least one or more modulators inhibit expression and/or function of stromal derived factor-1 (SDF-1), chemokine receptor 4 (CXCR4), or both stromal derived factor-1 (SDF-1) and chemokine receptor 4 (CXCR4) expression and/or function.

5. A method of preventing or treating pulmonary hypertension comprising:
administering to a patient a therapeutically effective dose of a composition comprising at least one modulator of: stromal derived factor-1 (SDF-1), chemokine receptor 4 (CXCR4), or stromal derived factor-1 (SDF- 1) and chemokine receptor 4 (CXCR4), or combinations thereof, in a therapeutically effective dose in a pharmaceutical carrier; and,
treating pulmonary hypertension.

6. The method of claim 5, wherein the at least one modulator inhibits expression and/or function of stromal derived factor-1 (SDF-1) and/or receptors thereof.

7. The method of claim 5, wherein the one or more modulators inhibit expression and/or function of chemokine receptor 4 (CXCR4) and/or ligands thereof.

8. The method of claim 5, wherein the one or more modulators inhibit stromal derived factor-1 (SDF-1) and chemokine receptor 4 (CXCR4).

9. The method of claim 5, wherein said modulator comprising: nucleic acids, oligonucleotides, polynucleotides, peptides, polypeptides, antibodies, aptamers, small molecules, organic molecules, inorganic molecules or combinations thereof.

10. The method of claim 9, wherein said modulator comprising: an aptamer specific for SDF-1, anti-SDF-1 antibody, or fragments thereof.

11. The method of claim 9, wherein said modulator comprising: an aptamer specific for CXCR4, an anti-CXCR4 antibody, or antagonist.

12. The method of claim 5, wherein the modulation of SDF-1 and/or CXCR4 decreases stem cell mobilization and/or stem cell recruitment into lungs and ventricles as compared to normal controls.

13. The method of claim 12, wherein the stem cells comprise c-kit⁺, Sca-1⁺ and/or IsI-1⁺ cells.

14. A method of decreasing stem cell mobilization or recruitment to an organ in vivo comprising:
administering to a patient a therapeutically effective dose of at least one modulator of: stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier; and,
decreasing stem cell mobilization or recruitment to an organ in vivo.

15. The method of claim 14, wherein the one or more modulators inhibit expression and/or function of stromal derived factor-1 (SDF-1) and/or receptors thereof.

16. The method of claim 14, wherein the modulator inhibits expression and/or function of chemokine receptor 4 (CXCR4) and/or ligands thereof.

17. The method of claim 14, wherein the one or more modulators inhibit stromal derived factor-1 (SDF-1) and chemokine receptor 4 (CXCR4).

18. The method of claim 14, wherein said modulator comprising nucleic acids, oligonucleotides, polynucleotides, peptides, polypeptides, antibodies, aptamers, small molecules, organic molecules, inorganic molecules or combinations thereof.

19. The method of claim 18, wherein said modulator comprising an aptamer, anti-SDF-1 antibody, fragments, peptides, antisense oligonucleotides, or combinations thereof.

20. The method of claim 18, wherein said modulator comprising an aptamer, anti-CXCR4 antibody, antagonists, peptides, antisense oligonucleotides, or combinations thereof.

21. The method of claim 14, wherein the modulation of SDF-1 and/or CXCR4 decreases stem cell mobilization and/or stem cell recruitment into lungs and ventricles as compared to normal controls.

22. The method of claim 21, wherein the stem cells comprise c-kit⁺, Sca-1⁺ and/or IsI-1⁺ cells.

23. A method of modulating SDF-1/CXCR4 signaling pathway in vitro or in vivo comprising:
contacting a cell, organ or tissue, or administering to a patient a therapeutically effective dose of at least one modulator of: stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier; and,
modulating the SDF-1/CXCR4 signaling pathway.

24. The method of claim 23, wherein the at least one modulator inhibits expression and/or function of stromal derived factor-1 (SDF-1) and/or receptors thereof.

25. The method of claim 23, wherein the at least one modulator inhibits expression and/or function of chemokine receptor 4 (CXCR4) and/or ligands thereof.

26. The method of claim 23, wherein the at least one or more modulators inhibit stromal derived factor-1 (SDF-1) and chemokine receptor 4 (CXCR4).

27. The method of claim 23, wherein said modulator comprising: nucleic acids, oligonucleotides, polynucleotides, peptides, polypeptides, antibodies, aptamers, small molecules, organic molecules, inorganic molecules or combinations thereof.

28. The method of claim 27, wherein said modulator comprising an aptamer, anti-SDF-1 antibody, fragments, peptides, antisense oligonucleotides, or combinations thereof.

29. The method of claim 27, wherein said modulator comprising an aptamer, anti-CXCR4 antibody, antagonists, peptides, antisense oligonucleotides, or combinations thereof.

30. The method of claim 23, wherein the modulation of SDF-1 and/or CXCR4 decreases stem cell mobilization and/or stem cell recruitment into lungs and ventricles as compared to normal controls.

31. The method of claim 30, wherein the stem cells comprise c-kit⁺, Sca-1⁺ and/or IsI-1⁺ cells.

32. A method of preventing or reversing hypoxia-induced pulmonary vascular remodeling in vivo comprising:
administering to a patient a therapeutically effective dose of at least one modulator of: stromal derived factor-1 (SDF-1), chemokine receptor 4 (CXCR4), stromal derived factor-1 (SDF-1) and chemokine receptor 4 (CXCR4), or combinations thereof, in a therapeutically effective dose in a pharmaceutical carrier; and,
preventing or reversing hypoxia-induced pulmonary vascular remodeling in vivo.

33. The method of claim 32, wherein the modulator inhibits expression and/or function of stromal derived factor-1 (SDF-1) and/or receptors thereof.

34. The method of claim 32, wherein the modulator inhibits expression and/or function of chemokine receptor 4 (CXCR4) and/or ligands thereof.

35. The method of claim 32, wherein one or more modulators inhibit stromal derived factor-1 (SDF-1) and chemokine receptor 4 (CXCR4).

36. The method of claim 32, wherein said modulator comprising: nucleic acids, oligonucleotides, polynucleotides, peptides, polypeptides, antibodies, aptamers, small molecules, organic molecules, inorganic molecules or combinations thereof.

37. The method of claim 36, wherein said modulator comprising an aptamer, anti-SDF-1 antibody, fragments, peptides, antisense oligonucleotides, or combinations thereof.

38. The method of claim 36, wherein said modulator an aptamer, anti-CXCR4 antibody, antagonists, peptides, antisense oligonucleotides, or combinations thereof.

39. The method of claim 32, wherein the modulation of SDF-1 and/or CXCR4 decreases stem cell mobilization and/or stem cell recruitment into lungs and ventricles as compared to normal controls.

40. The method of claim 39, wherein the stem cells comprise c-kit⁺, Sca-1⁺ and/or IsI-1⁺ cells.

41. A method of preventing and treating disorders associated with hypoxia comprising:
administering to a patient a therapeutically effective dose of at least one modulator of stromal derived factor-1 (SDF-1), chemokine receptor 4 (CXCR4), or stromal derived factor-1 (SDF-1) and chemokine receptor 4 (CXCR4), or combinations thereof, in a therapeutically effective dose in a pharmaceutical carrier; and,
preventing or treating disorders associated with hypoxia.

42. A method of identifying candidate agents which modulate stromal derived factor-1 (SDF-1) and/or chemokine receptor 4 (CXCR4) expression and/or function comprising:
obtaining a stromal derived factor-1 (SDF-1) molecule, chemokine receptor 4 (CXCR4) molecule or cell expressing at least one of these molecules;
contacting said molecules or cell expressing at least one of these molecules with a candidate agent;
measuring expression and/or function of SDF-1 and CXCR4 as compared to a normal control; and, identifying candidate agents which modulate stromal derived factor-1 (SDF-1) and/or chemokine receptor 4 (CXCR4) expression and/or function.
